(19) United States
(12) Patent Application Publication
(10) Pub. No.: US 2012/0237446 A1
(43) Pub. Date: Sep. 20, 2012

(54) Compounds for binding and imaging amyloid plaques and their use

(75) Inventors: Ulrike Röhn, Berlin (DE); Hermann Künzer, Berlin (DE); Tobias Heinrich, Berlin (DE); Markus Berger, Berlin (DE); Damian Brockschnieder, Haan (DE); Sabine Krause, Berlin (DE); Thomas Dyrks, Berlin (DE); Andrea Thiele, Bernau (DE); Ursula Mönning, Woltersdorf (DE); Ulf Bömer, Glienicke (DE)

(73) Assignee: BAYER PHARMA AKTIENGESELLSCHAFT, Berlin (DE)

(21) Appl. No.: 13/429,667

(22) Filed: Mar. 26, 2012

(30) Foreign Application Priority Data
Mar. 25, 2005 (EP) 11159789

(51) Int. Cl. A61K 31/137; C07C 255/58; A61K 31/277; C07D 213/61; G01N 33/53; C07C 309/69; A61K 31/255; A61K 51/04; A61P 25/28; C07C 225/22; A61K 31/44

(52) U.S. Cl. 424/1.89; 564/342; 514/654; 558/415; 514/524; 546/315; 514/355; 558/51; 514/517; 435/7.1

(57) ABSTRACT

The present invention relates to novel compounds (3-aminopropen-1-ones) useful for binding and imaging beta amyloid deposits and their use in detecting or treating Alzheimer's disease and amyloidosis.

The present invention relates to novel compounds (3-aminopropen-1-ones) useful for binding and imaging amyloid deposits and their use in detecting or treating Alzheimer's disease and amyloidosis.

### BACKGROUND OF THE INVENTION

Alzheimer's disease (AD) is a progressive neurodegenerative disorder marked by loss of memory, cognition and behavioral stability. AD is defined pathologically by extracellular plaques comprised of fibrillar deposits of the beta amyloid (Aβ) peptide and neurofibrillary tangles comprised of paired helical filaments of hyperphosphorylated tau. The 39 to 43 amino acids comprising Aβ peptides are derived from the larger amyloid precursor protein (APP). In the amyloidogenic pathway, Aβ peptides are cleaved from APP by the sequential proteolysis by β- and γ-secretases. Aβ peptides are released as soluble proteins and can be detected at low levels in the cerebrospinal fluid (CSF) in normal aging brains. During the progress of AD the Aβ peptides aggregate and form amyloid deposits in the parenchyma and vasculature of the brain, which can be detected post mortem as diffuse and senile plaques and vascular amyloid during histological examination (for a recent review see: Blennow et al., Lancet. 2006, 368(9533): 387-403).

Alzheimer's disease is becoming a great health and social economical problem all over the world. There are great efforts being made to develop techniques and methods for the early detection and effective treatment of the disease. Currently, diagnosis of AD in an academic setting of memory-disorder clinics is approximately 85-90% accurate (Petrella J R et al., Radiology 2003, 226: 315-36). It is based on the exclusion of a variety of diseases causing similar symptoms and the careful neurological and psychiatric examination, as well as neuropsychological testing. However, post mortem histological examination of the brain is still the only definite diagnosis of this disease. Thus the in vivo detection of one pathological feature of the disease—the deposition of amyloid aggregates in the brain—is thought to have a big impact on the early detection of AD and differentiation from other dementias. Additionally, most disease modifying therapies that are under development are aiming at lowering the amyloid load in the brain. Thus imaging the amyloid load in the brain may provide an essential tool for patient stratification and treatment monitoring.

In addition, amyloid deposits are also known to play a role in amyloidosis, in which amyloid proteins are abnormally deposited in different organs and/or tissues, causing disease. For a recent review see Chiti et al., Annu. Rev. Biochem. 2006, 75: 333-66. Potential ligands for visualizing amyloid aggregates in the brain must show a high binding affinity to amyloid and must cross the blood brain barrier. PET tracers that have been already investigated in humans regarding their binding patterns in brains of AD patients are [¹⁸F]FDDNP (Shoghi-Jadid et al., Am. J. Geriatric Psychiatry 2002, 10: 24-35), [¹¹C]PIB (Klunk et al., Ann. Neurol. 2004, 55: 306-319), [¹¹C]SB-13 (Verhoeff et al., Am. J. Geriatric Psychiatry 2004, 12: 584-595; [¹⁸F]Bay 94-9172 (Rowe et al., Lancet Neurol. 2008, 7: 129-135), [¹¹C]13F227 (Kudo et al., J. Nucl. Med. 2007, 49: 554-561), and [¹⁸F]PIB (Nelissen et al., J. Nucl. Med. 2009, 50(8):1251-1259). For recent reviews see Lockhardt, Drug Discov. Today 2006, 11: 1093-1099; Henriksen et al., Eur. J. Nucl. Med. Mol. Imaging. 2008, 35 Suppl. 1: 75-81; Cohen, Mol. Imaging. Biol. 2007, 9: 204-216; Nordberg, Curr. Opin. Biol. 2007, 20: 398-402; Small et al., Neurology 2008, 7: 161-172; Nordberg et al., Nat. Rev. Neurol. 2010, 6(2): 78-87.

Besides their specific binding to amyloid deposits in the brain, the currently most promising PET tracers show a disadvantageous non-specific accumulation, especially in white matter brain regions in AD patients as well as in healthy controls (HC). Generally, non-specific background binding interferes with the image quality and could e.g. impair the quantification of amyloid and the diagnosis of very early stages of the disease.

### Problem to be Solved and its Solution

The problem underlying the present invention was to provide compounds suited for detecting amyloid deposits in patients with amyloid-related diseases with high specificity at an early stage of the disease.

Surprisingly it was observed that special [¹⁸F] labeled 3-amino-propenones can be used as diagnostic agent or PET imaging agent for imaging diseases associated with deposits of beta amyloid (Aβ). As will be shown herein, the special 3-amino-propenones can penetrate the intact blood-brain barrier and bind specifically to beta amyloid deposits. The present invention provides novel tracers with high affinity for beta amyloid (Aβ) and rapid elimination of the unspecifically bound tracer from the brain.

There are 3-amino-propenones disclosed in prior art. However, they have not been disclosed for use as diagnostic agent or PET imaging agent for imaging diseases associated with altered expression of beta amyloid (Aβ) peptide. Mesogenic 3-amino-propenones are disclosed in Mol. Cryst. Liq. Cryst. 1993, 237: 75-84. US2009/0163545A1 discloses 3-amino-propenones and their use for altering the lifespan of a eukaryotic organism.

Hence, the superior fitness of the 3-amino-popenones of the present invention as disclosed hereinafter could not be expected by the person skilled in the art.

### SUMMARY OF THE INVENTION

The present invention relates to the use of compounds of general formula I as diagnostic agent or imaging agent for imaging diseases associated with beta amyloid (Aβ) deposits

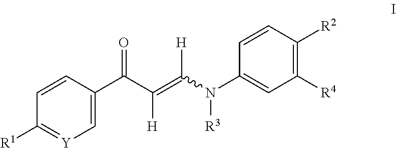

wherein
R¹ is selected from the group consisting of H, phenyl, and X;
R² is selected from the group consisting of H, OCH₃, N(CH₃)₂, O—(CH₂)ₙ—X; (CH₂)ₙ—X, and X;
R³ is H;
R⁴ is selected from the group consisting of H and Br;
X is selected from the group consisting of F and ¹⁸F;
Y is selected from the group consisting of CH, CCN, CCF₃ and N; and
n is 1, 2 or 3;
   with the proviso that one of the substituents R¹ or R² comprises ¹⁸F or is ¹⁸F;
   including all isomeric forms of said compound, including but not limited to E/Z isomers, as well as mixtures of E/Z isomers, and any pharmaceutically acceptable salt or complex thereof.

The present invention also relates to novel compounds of formula II

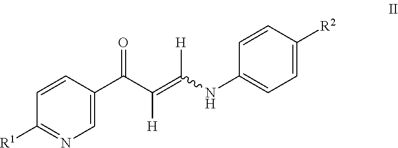

wherein
R¹ is F or ¹⁸F,
R² is selected from the group consisting of H, OCH₃, and N(CH₃)₂;
   including all isomeric forms of said compound, including but not limited to E/Z isomers, as well as mixtures of E/Z isomers, and any pharmaceutically acceptable salt or complex thereof.

The compounds of formula II selectively bind to beta amyloid (Aβ). The [¹⁸F] labelled compounds can be used as a tracer for PET imaging. The fluoro analogues can be used as reference standards.

The present invention also relates to compounds of formula III

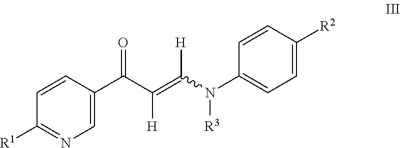

wherein
R¹ is selected from the group consisting of F, Cl, Br, I, NO₂, N⁺(CH₃)₃, I⁺aryl, and S⁺aryl₂;
R² is selected from the group consisting of H, OCH₃, and N(CH₃)₂; and
R³ is H or C(O)OC(CH₃)₃;
   including all isomeric forms of said compound, including but not limited to E/Z isomers, as well as mixtures of E/Z isomers.

Compounds of formula III can be used as precursors for making compounds of formula II.

The present invention also relates to processes for making compounds of formulae II and III.

The present invention also relates to a pharmaceutical composition comprising a compound of formula I, II or III, and to a kit comprising a compound or a composition according to the invention.

### DETAILED DESCRIPTION OF THE INVENTION

The present invention is directed to compounds that bind to amyloid deposits and are able to pass the blood-brain barrier, and are therefore useful in diagnosing Alzheimer's disease and amyloidosis in a patient, preferably at an early stage of the disease.

In a first aspect, the invention is directed to the use of compounds of general formula I as diagnostic agent or imaging agent for imaging diseases associated with beta amyloid (Aβ) deposits

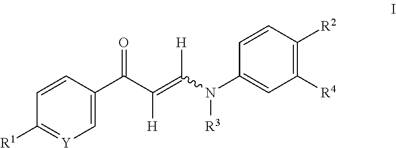

wherein
R¹ is selected from the group consisting of H, phenyl or X;
R² is selected from the group consisting of H, OCH₃, N(CH₃)₂, O—(CH₂)ₙ—X; (CH₂)ₙ—X or X;
R³ is H;
R⁴ is selected from the group consisting of H or Br;
X is selected from the group consisting of F or ¹⁸F;
Y is selected from CH, CCN, CCF₃ or N; and
n is 1, 2 or 3;
   with the proviso that one of the substituents R¹ and R² comprises ¹⁸F or is ¹⁸F;
   including all isomeric forms of said compound, including but not limited to E/Z isomers, as well as mixtures of E/Z isomers, and any pharmaceutically acceptable salt or complex thereof.

Preferably, the compound is used as an imaging tracer or radiopharmaceutical agent for imaging diseases associated with beta amyloid (Aβ) deposits.

The radiopharmaceutical imaging tracer/agent is a Positron Emission Tomography (PET) suitable radiopharmaceutical imaging tracer/agent.

In other words, the invention is directed to the use of a compound of formula I for the manufacture of a radiopharmaceutical imaging tracer/agent for imaging diseases associated with beta amyloid deposits.

In other words, the invention is directed to a compound of general formula I for use in the imaging of beta amyloid deposits.

The invention is also directed to a method for imaging or diagnosing of diseases associated with beta amyloid deposits comprising the steps:
administering to a mammal an effective amount of a composition comprising a compound of formula I,
obtaining PET images of the mammal and
assessing PET images.

Preferably, mammal is human.

The administration of the compounds or pharmaceutical compositions according to the invention is performed in any of the generally accepted modes of administration available in the art. Intravenous deliveries are preferred.

Preferably, the compounds or compositions according to the invention are administered such that the dose of the active compound for imaging is in the range of 37 MBq (1 mCi) to 740 MBq (20 mCi). In particular, a dose in the range from 100 MBq to 400 MBq will be used.

Formula I is directed to the E isomer as well as to the Z isomer.

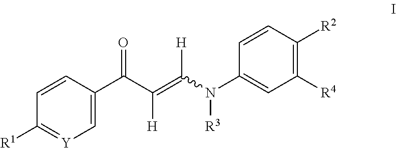

So, formula I is a general formula, representing formulae Ia (E isomer) and Ib (Z isomer):

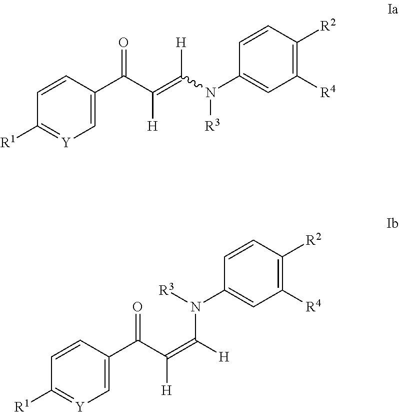

A compound of formula I therefore includes the E isomer, the Z isomer, and any mixture of E/Z isomers.

Furthermore, a compound of formula I includes pharmaceutically acceptable salts or complexes thereof.

Pharmaceutically acceptable salts of the compounds according to the invention include salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalene disulfonic acid, acetic acid, trifluoroacetic acid, propionic acid, lactic acid, tartaric acid, malic acid, citric acid, fumaric acid, maleic acid and benzoic acid.

Pharmaceutically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

The term “alkyl” as used in this specification represents a linear or branched alkyl radical having, as a rule, 1 to 6, preferably 1 to 4, especially preferably 1 to 3, carbon atoms, by way of example and by preference methyl, ethyl, n-propyl, iso-propyl, tert-butyl, n-pentyl and n-hexyl.

The term “aryl” as used in this specification is to be understood as preferably meaning a monovalent, aromatic or partially aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a biphenyl group, or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a ring having 13 carbon atoms, (a “C₁₃-aryl” group), e.g. a fluorenyl group, or a ring having 14 carbon atoms, (a “C₁₄-aryl” group), e.g. an anthranyl group.

In a preferred embodiment, the compound of formula I is selected from the group of compounds consisting of
(E/Z)-3-{[4-([¹⁸F]fluoromethoxy)phenyl]amino}-1-phenylprop-2-en-1-one

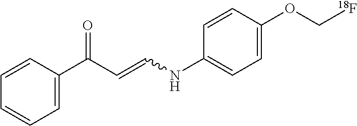

(E/Z)-3-{[4-(2-[¹⁸F]fluoroethoxy)phenyl]amino}-1-phenylprop-2-en-1-one

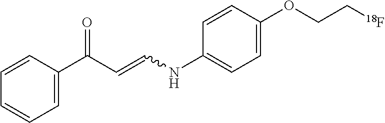

(E/Z)-1-(4-Bromophenyl)-3-{[4-(2-[¹⁸F]fluoroethoxy)phenyl]amino}prop-2-en-1-one

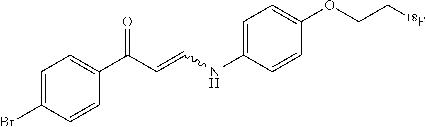

(E/Z)-3-{[4-(3-[¹⁸F]fluoropropoxy)phenyl]amino}-1-phenylprop-2-en-1-one

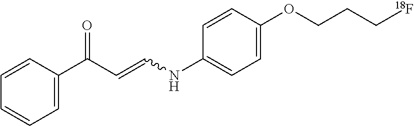

(E/Z)-1-(4-[¹⁸F]fluorophenyl)-3-[(4-methoxyphenyl)amino]prop-2-en-1-one

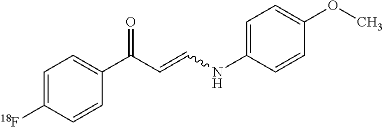

(E/Z)-3-{[4-(Dimethylamino)phenyl]amino}-1-(4-[¹⁸F]fluorophenyl)prop-2-en-1-one

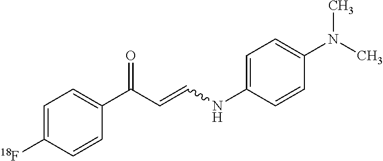

(E/Z)-3-{[4-(Dimethylamino)phenyl]amino}-1-[4-[¹⁸F]fluoro-3-(trifluoromethyl)phenyl]prop-2-en-1-one

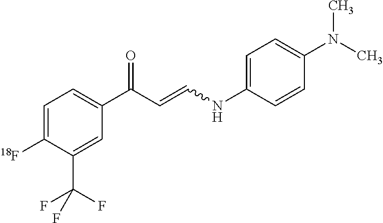

5-[(E/Z)-3-{[4-(Dimethylamino)phenyl]amino}acryloyl]-2-[¹⁸F]fluorobenzonitrile

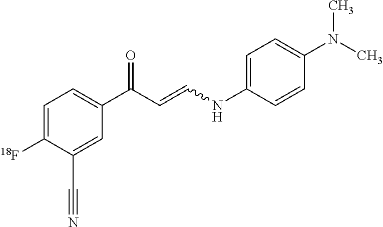

(E/Z)-1-[4-[¹⁸F]fluoro-3-(trifluoromethyl)phenyl]-3-[(4-methoxyphenyl)amino]prop-2-en-1-one

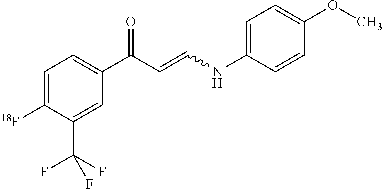

(E/Z)-3-{[4-(Dimethylamino)phenyl]amino}-1-(6-[¹⁸F]fluoropyridin-3-yl)prop-2-en-1-one

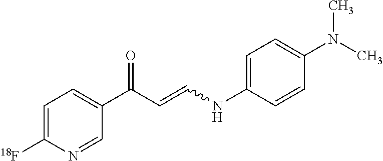

(E/Z)-1-(6-[¹⁸F]fluoropyridin-3-yl)-3-[(4-methoxyphenyl)amino]prop-2-en-1-one

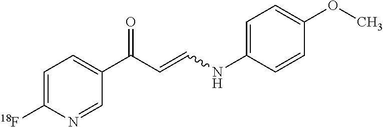

(E/Z)-3-[(4-[¹⁸F]-fluorophenyl)amino]-1-phenylprop-2-en-1-one

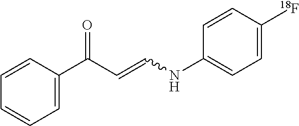

(E/Z)-3-[(3-Bromo-4-[¹⁸F]fluorophenyl)amino]-1-phenylprop-2-en-1-one

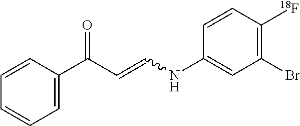

(E/Z)-1-Biphenyl-4-yl-3-{[4-(2-[¹⁸F]fluoroethoxy)phenyl]amino}prop-2-en-1-one

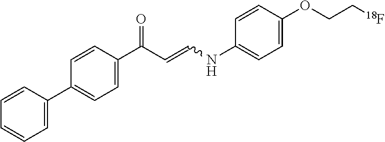

(E/Z)-3-{[4-(3-[¹⁸F]fluoropropyl)phenyl]amino}-1-phenylprop-2-en-1-one

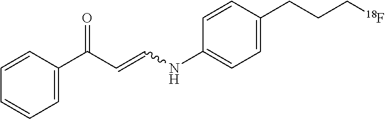

(E/Z)-3-{[4-(Dimethylamino)phenyl]amino}-1-(6-[¹⁸F]-fluoropyridin-3-yl)prop-2-en-1-one

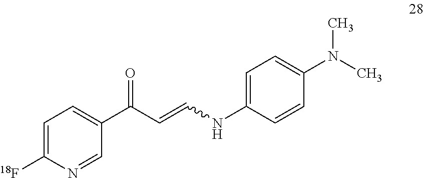

including all isomeric forms of said compounds, including but not limited to E/Z isomers, as well as mixtures of E/Z isomers, and any pharmaceutically acceptable salt or complex thereof.

In another preferred embodiment, the compound of formula I is selected from the group of compounds of formula IIa

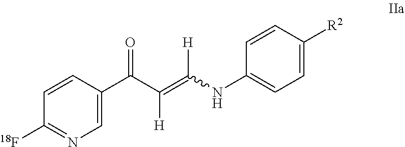

wherein R² is selected from the group consisting of H, OCH₃, and N(CH₃)₂;
including all isomeric forms of said compounds, including but not limited to E/Z isomers, as well as mixtures of E/Z isomers, and any pharmaceutically acceptable salt or complex thereof.

In a second aspect, the invention is directed to novel compounds of formula I per se,

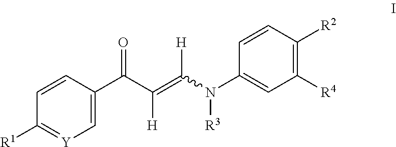

wherein
R¹ is F or ¹⁸F;
R² is selected from the group consisting of H, OCH₃, or N(CH₃)₂;
R³ is H;
R⁴ is H; and
Y is N;
   including all isomeric forms of said compound, including but not limited to E/Z isomers, as well as mixtures of E/Z isomers, and any pharmaceutically acceptable salt or complex thereof.

In other words, the invention is directed to compounds of formula II

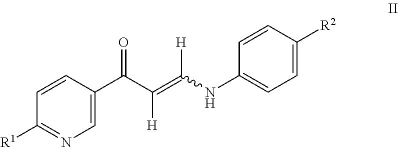

wherein
R¹ is F or ¹⁸F;
R² is selected from the group consisting of H, OCH₃ or N(CH₃)₂;
   including all isomeric forms of said compound, including but not limited to E/Z isomers, as well as mixtures of E/Z isomers, and any pharmaceutically acceptable salt or complex thereof.

In a preferred embodiment, the compound of formula II is selected from the group of compounds consisting of
(E/Z)-3-{[4-(Dimethylamino)phenyl]amino}-1-(6-fluoropyridin-3-yl)prop-2-en-1-one

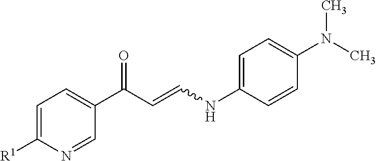

wherein R¹ is F or ¹⁸F_{;}
(E/Z)-1-(6-Fluoropyridin-3-yl)-3-[(4-methoxyphenyl)amino]prop-2-en-1-one

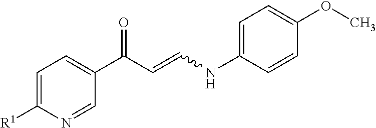

wherein R¹ is F or ¹⁸F_{;}
(E/Z)-3-{[4-(Dimethylamino)phenyl]amino}-1-(6-[¹⁸F]fluoropyridin-3-yl)prop-2-en-1-one

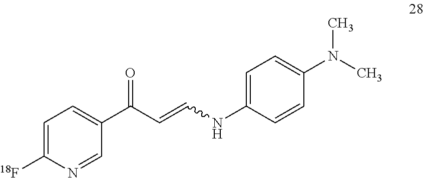

including all isomeric forms of said compounds, including but not limited to E/Z isomers, as well as mixtures of E/Z isomers, and any pharmaceutically acceptable salt or complex thereof.

In a third aspect, the invention is directed to compounds according to formula III,

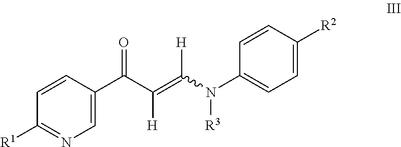

wherein
R¹ is selected from the group consisting of F, Cl, Br, I, NO₂, N⁺(CH₃)₃, I⁺aryl, and S⁺aryl_{2;}
R² is selected from the group consisting of H, OCH₃, and N(CH₃)₂; and
R³ is H or C(O)OC(CH₃)₃;
   including all isomeric forms of said compound, including but not limited to E/Z isomers, as well as mixtures of E/Z isomers.

Compounds of formula III can be used as precursors for the preparation of ¹³F labelled compounds of formula II.

In a preferred embodiment of the present invention, the compound of formula III is selected from the group of compounds consisting of
(E/Z)-1-(6-Bromopyridin-3-yl)-3-{[4-(dimethylamino)phenyl]amino}prop-2-en-1-one

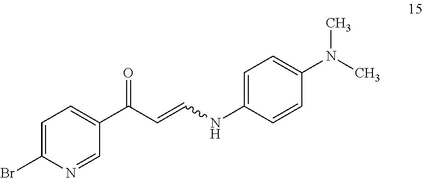

(E/Z)-1-(6-Chloropyridin-3-yl)-3-{[4-(dimethylamino)phenyl]amino}prop-2-en-1-one

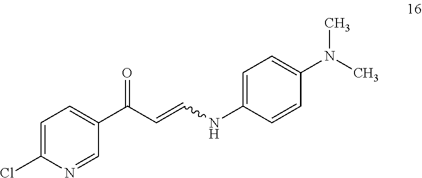

(E/Z)-3-{[4-(Dimethylamino)phenyl]amino}-1-(6-nitropyridin-3-yl)prop-2-en-1-one

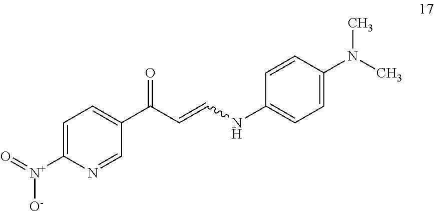

(E/Z)-3-{[4-(Dimethylamino)phenyl]amino}-1-(6-iodopyridin-3-yl)prop-2-en-1-one

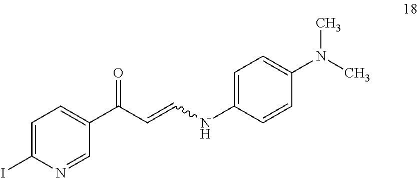

tert-Butyl N-[4-(dimethylamino)phenyl]-N-[(E/Z)-3-(6-nitropyridin-3-yl)-3-oxoprop-1-en-1-yl]carbamate

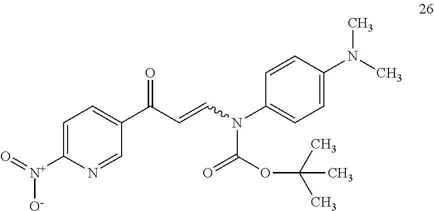

tert-Butyl N-[4-(dimethylamino)phenyl]-N-[(E/Z)-3-(6-iodopyridin-3-yl)-3-oxoprop-1-en-1-yl]carbamate

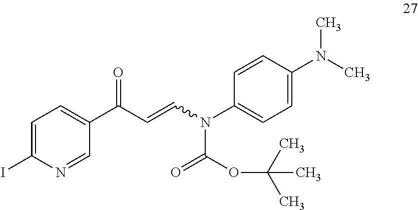

including all isomeric forms of said compounds, including but not limited to E/Z isomers, as well as mixtures of E/Z isomers.

In a fourth aspect, the invention is directed to methods of preparing compounds of the general formula III.

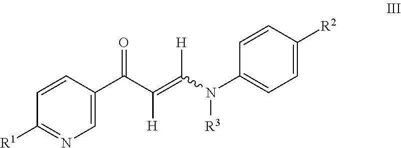

wherein
R¹ is selected from the group consisting of F, Cl, Br, I, NO₂, N⁺(CH₃)₃, I⁺aryl, and S⁺aryl_{2;}
R² is selected from the group consisting of H, OCH₃, and N(CH₃)₂; and
R³ is H;

The method comprises the steps
providing a compound of formula IV;

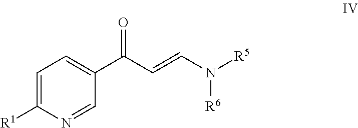

wherein
R¹ is selected from the group consisting of F, Cl, Br, I, NO₂, N⁺(CH₃)₃, I⁺aryl, and S⁺aryl₂;
R⁵, R⁶ independently are selected from the group consisting of H and alkyl; preferably R⁵ and R⁶ are methyl;
providing a compound of formula V;

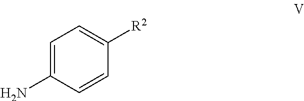

R² is selected from the group consisting of H, OCH₃, and N(CH₃)₂;
   reacting the compound of formula IV with the compound of formula V to get the compound of formula III.

In a fifth aspect, the invention is directed to a method of preparing a compound of formula IIa

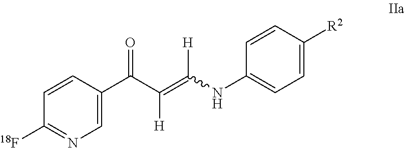

wherein
R² is selected from the group consisting of H, OCH₃, and N(CH₃)₂.

The method comprises the steps
providing a compound of formula III

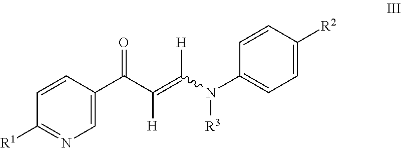

wherein
   R¹ is selected from the group consisting of F, Cl, Br, I, NO₂, N⁺(CH₃)₃, I⁺aryl, and S⁺aryl_{2;}
   R² is selected from the group consisting of H, OCH₃, and N(CH₃)₂; and
   R³ is H or C(O)OC(CH₃)₃;
reacting the compound of formula III with a ¹⁸F-fluorination agent;
optionally replacing the C(O)OC(CH₃)₃ group by an H atom, if R³ is C(O)OC(CH₃)₃.

The ¹⁸F-fluorination agent is a reagent containing ¹⁸F and being suitable for substituting the R¹ group in the compound of formula III by an ¹⁸F atom.

In a preferred embodiment [¹⁸F]fluoride/TBAOH (TBAOH=tetrabutylammonium hydroxide) and a solvent is used as a ¹⁸F-fluorination agent.

In a preferred embodiment, the ¹⁸F-fluorination agent is 4,7,13,16,21,24-Hexaoxa-1,10 diazabicyclo[8.8.8]-hexacosane K¹⁸F (crownether salt Kryptofix K¹⁸F), K¹⁸F, H¹⁸F, KH¹⁸F₂ or tetraalkylammonium salt of ¹⁸F. More preferably, the fluorination agent is K¹⁸F, H¹⁸F, or KH¹⁸F₂.

The solvents used can be N,N-Dimethylformamide (DMF), Dimethylsulfoxide (DMSO), Acetonitrile (CH₃CN), N,N-Dimethylacetamide (DMA) etc., preferably DMSO, CH₃CN or DMF. The solvents can also be a mixture of solvents as indicated above.

In a sixth aspect, the invention is directed to a composition comprising a compound of formula I and at least one physiologically acceptable carrier, diluent, adjuvant or excipient.

The adjuvants include, inter alia, carriers, solvents, and/or stabilizers.

The person skilled in the art is familiar with adjuvants which are suitable for the desired pharmaceutical formulations, preparations or compositions on account of his/her expert knowledge.

As used herein, the term “carrier” refers to e.g. microcrystalline cellulose, lactose, mannitol.

As used herein, the term “solvents” refers to e.g. liquid polyethylene glycols, ethanol, corn oil, cottonseed oil, glycerol, isopropanol, mineral oil, oleic acid, peanut oil, purified water, water for injection, sterile water for injection and sterile water for irrigation.

As used herein, the term “stabilizers” refers to e.g. antioxidants, such as, ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite.

In a first embodiment, the compound of formula I is selected from the group of compounds of formula IIa

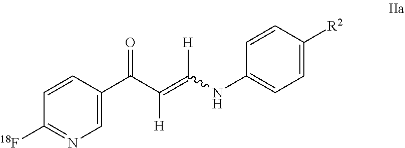

wherein R² is selected from the group consisting of H, OCH₃, and N(CH₃)₂.

A pharmaceutical composition comprising a compound of formula IIa can administered to a patient in order to image amyloid deposits. Thus, the composition can be used as a diagnostic agent or PET imaging agent.

In a second embodiment, the invention is directed to a composition comprising a compound of formula IIb

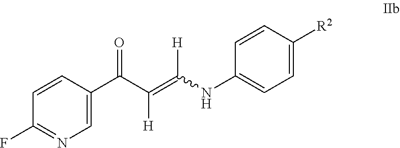

wherein R² is selected from the group consisting of H, OCH₃, and N(CH₃)₂.

Such composition can be used for analytical purposes.

In a third embodiment, the invention is directed to a composition comprising a compound of formula III

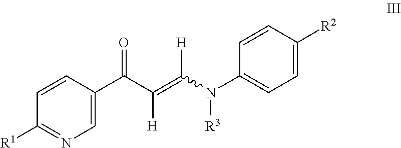

wherein
R¹ is selected from the group consisting of F, Cl, Br, I, NO₂, N⁺(CH₃)₃, I⁺aryl, and S⁺ aryl_{2;}
R² is selected from the group consisting of H, OCH₃, and N(CH₃)₂; and
R³ is H or C(O)OC(CH₃)₃;

Such composition can be used for manufacturing of a compound of formula IIa and/or IIb and for analytical purposes.

In a seventh aspect, the present invention is directed to a kit comprising at least one sealed vial containing a predetermined quantity of a compound of formulae I, II, IIa, IIb and/or III. The compounds of general formulae I, II, IIa, IIb and III are herein defined as above and encompass all embodiments and preferred features.

Optionally the kit comprises a pharmaceutically acceptable carrier, diluent, excipient or adjuvant.

Preferably, the kit comprises predefined quantity of compound of formula IIb or III, and one or more solid-phase extraction cartridges/columns for the purification of compound of formula IIa.

The compounds of general formula I, IIa, and IIb are herein defined as above and encompass all embodiments and preferred features.

Preferably, the kit comprises physiologically acceptable vehicle or carrier and optional adjuvants and preservatives, reagents suitable to perform the herein disclosed reactions and/or methods to generate the [¹⁸F] labeling reagents.

Furthermore, the kit may contain instructions for its use.

In an eighth aspect, the invention is directed to the use of compounds of general formulae I, IIa or IIb for chromatographic identification and as radioactivley labelled tools in in vitro binding assays for amyloidogenic aggregates. As a radioactive label for example tritium can be introduced into the molecules by standard tritiation reactions that are known to an expert in the field.

Compounds of general formula IIb are useful as references and/or measurement agents. The compounds of general formulae I, IIa, and IIb are herein defined as above and encompass all embodiments and preferred features.

Furthermore, the invention is related to:

1. A Compound of formula I

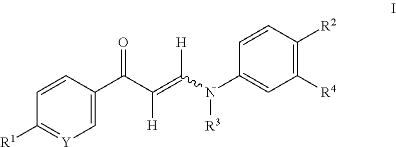

as diagnostic agent or imaging agent for imaging diseases associated with beta amyloid deposits
wherein
R¹ is selected from the group consisting of H, phenyl or X;
R² is selected from the group consisting of H, OCH₃, N(CH₃)₂, O—(CH₂)ₙ—X; (CH₂)ₙ—X or X_{;}
R³ is H;
R⁴ is selected from the group consisting of H or Br;
X is selected from the group consisting of F or ¹⁸F_{;}
Y is selected from CH, CCN, CCF₃ or N; and
n is 1, 2 or 3;
   with the proviso that one of the substituents R¹ and R² comprises ¹⁸F or is ¹⁸F;
   including all isomeric forms of said compound, including but not limited to E/Z isomers, as well as mixtures of E/Z isomers, and any pharmaceutically acceptable salt or complex thereof.
   2. A compound according to count 1, wherein the compound is selected from the group of compounds consisting of
(E/Z)-3-{[4-([¹⁸F]-fluoromethoxy)phenyl]amino}-1-phenylprop-2-en-1-one

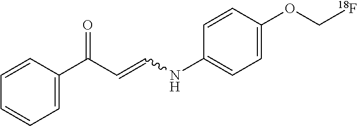

(E/Z)-3-{[4-(2-[¹⁸F]fluoroethoxy)phenyl]amino}-1-phenylprop-2-en-1-one

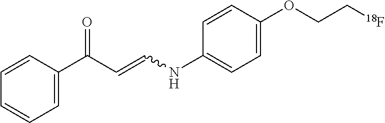

(E/Z)-1-(4-Bromophenyl)-3-{[4-(2-[¹⁸F]fluoroethoxy)phenyl]amino}prop-2-en-1-one

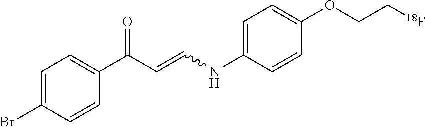

(E/Z)-3-{[4-(3-[¹⁸F]fluoropropoxy)phenyl]amino}-1-phenylprop-2-en-1-one

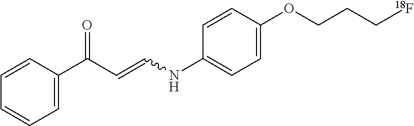

(E/Z)-1-(4-[¹⁸F]-fluorophenyl)-3-[(4-methoxyphenyl)amino]prop-2-en-1-one

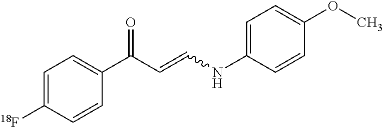

(E/Z)-3-{[4-(Dimethylamino)phenyl]amino}-1-(4-[¹⁸F]fluorophenyl)prop-2-en-1-one

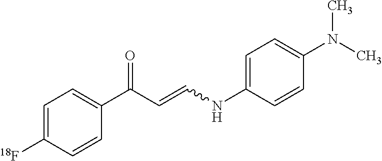

(E/Z)-3-{[4-(Dimethylamino)phenyl]amino}-1-[4-[¹⁸F]fluoro-3-(trifluoromethyl)phenyl]prop-2-en-1-one

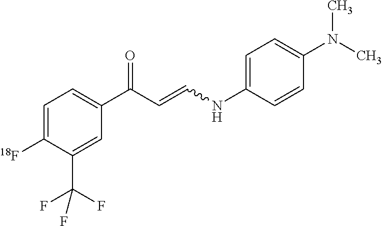

5-[(E/Z)-3-{[4-(Dimethylamino)phenyl]amino}acryloyl]-2-[¹⁸F]fluorobenzonitrile

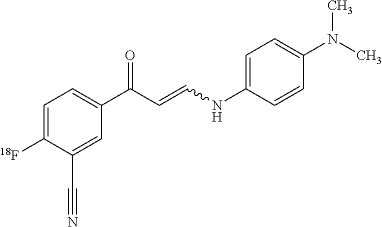

(E/Z)-1-[4-[¹⁸F]fluoro-3-(trifluoromethyl)phenyl]-3-[(4-methoxyphenyl)amino]prop-2-en-1-one

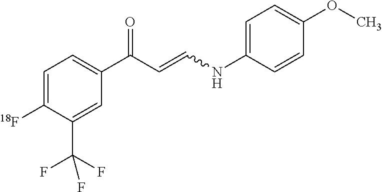

(E/Z)-3-{[4-(Dimethylamino)phenyl]amino}-1-(6-[¹⁸F]fluoropyridin-3-yl)prop-2-en-1-one

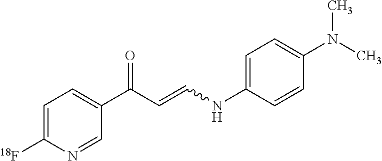

(E/Z)-1-(6-[¹⁸F]fluoropyridin-3-yl)-3-[(4-methoxyphenyl)amino]prop-2-en-1-one

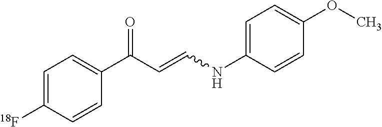

(E/Z)-3-[(4-[¹⁸F]fluorophenyl)amino]-1-phenylprop-2-en-1-one

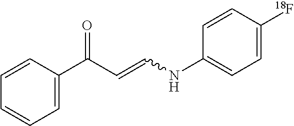

(E/Z)-3-[(3-Bromo-4-[¹⁸F]fluorophenyl)amino]-1-phenylprop-2-en-1-one

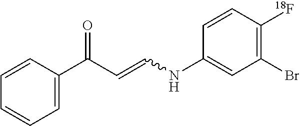

(E/Z)-1-Biphenyl-4-yl-3-{[4-(2-[¹⁸F]fluoroethoxy)phenyl}amino]prop-2-en-1-one

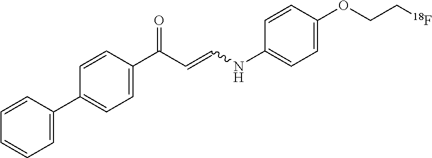

(E/Z)-3-{[4-(3-[¹⁸F]fluoropropyl)phenyl]amino}-1-phenylprop-2-en-1-one

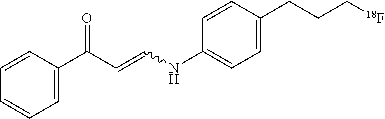

(E/Z)-3-{[4-(Dimethylamino)phenyl]amino}-1-(6-[¹⁸F]-fluoropyridin-3-yl)prop-2-en-1-one

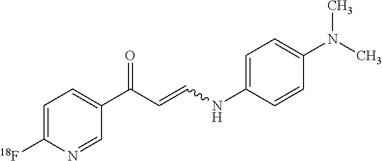

including all isomeric forms of said compounds, including but not limited to E/Z isomers, as well as mixtures of E/Z isomers, and any pharmaceutically acceptable salt or complex thereof.
3. A compound of formula II

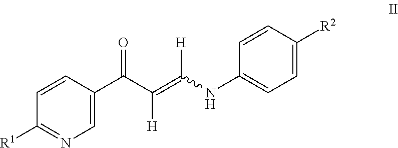

wherein
R¹ is F or ¹⁸F,
R² is selected from the group consisting of H, OCH₃ or N(CH₃)₂;
   including all isomeric forms of said compound, including but not limited to E/Z isomers, as well as mixtures of E/Z isomers, and any pharmaceutically acceptable salt or complex thereof.
   4. A compound according to count 3, wherein the compound is selected from the group of compounds consisting of
(E/Z)-3-{[4-(Dimethylamino)phenyl]amino}-1-(6-fluoropyridin-3-yl)prop-2-en-1-one

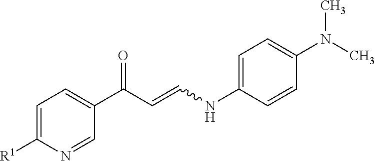

wherein R¹ is F or ¹⁸F_{;}
(E/Z)-1-(6-Fluoropyridin-3-yl)-3-[(4-methoxyphenyl)amino]prop-2-en-1-one

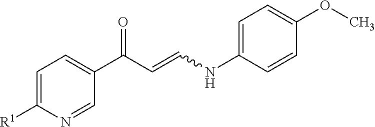

wherein R¹ is F or ¹⁸F_{;}
(E/Z)-3-{[4-(Dimethylamino)phenyl]amino}-1-(6-[¹⁸F]fluoropyridin-3-yl)prop-2-en-1-one

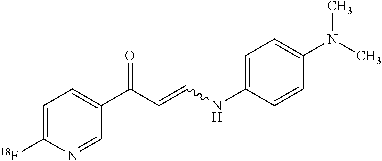

including all isomeric forms of said compound, including but not limited to E/Z isomers, as well as mixtures of E/Z isomers, and any pharmaceutically acceptable salt or complex thereof.
5. A compound of formula III

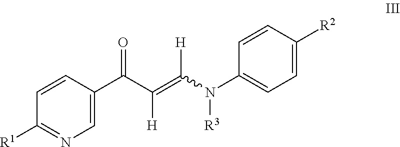

wherein
R¹ is selected from the group consisting of F, Cl, Br, I, NO₂, N⁺(CH₃)₃, I⁺aryl, and S⁺aryl_{2;}
R² is selected from the group consisting of H, OCH₃, and N(CH₃)₂; and
R³ is H or C(O)OC(CH₃)₃;
   including all isomeric forms of said compound, including but not limited to E/Z isomers, as well as mixtures of E/Z isomers.
   6. A compound according to count 5, wherein the compound is selected from the group of compounds consisting of
(E/Z)-1-(6-Bromopyridin-3-yl)-3-{[4-(dimethylamino)phenyl]amino}prop-2-en-1-one

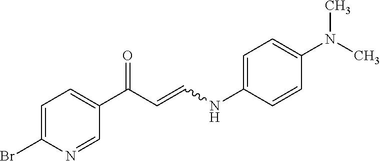

(E/Z)-1-(6-Chloropyridin-3-yl)-3-{[4-(dimethylamino)phenyl]amino}prop-2-en-1-one

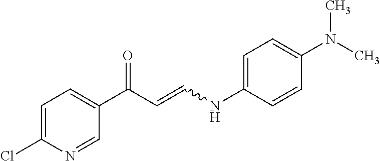

(E/Z)-3-{[4-(Dimethylamino)phenyl]amino}-1-(6-nitropyridin-3-yl)prop-2-en-1-one

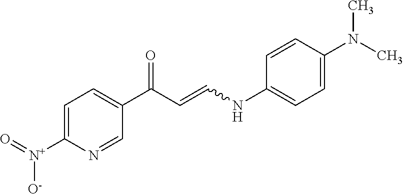

(E/Z)-3-{[4-(Dimethylamino)phenyl]amino}-1-(6-iodopyridin-3-yl)prop-2-en-1-one

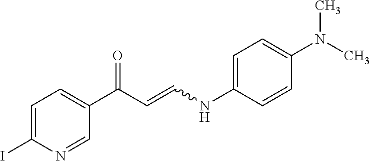

tert-Butyl N-[4-(dimethylamino)phenyl]-N-[(E/Z)-3-(6-nitropyridin-3-yl)-3-oxoprop-1-en-1-yl]carbamate

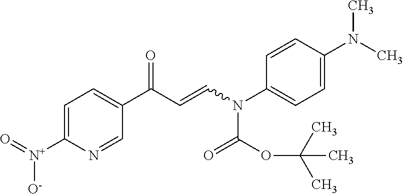

tert-Butyl N-[4-(dimethylamino)phenyl]-N-[(E/Z)-3-(6-iodopyridin-3-yl)-3-oxoprop-1-en-1-yl]carbamate

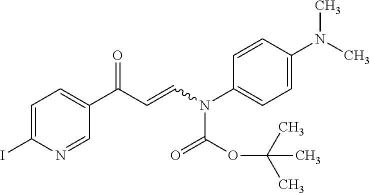

including all isomeric forms of said compounds, including but not limited to E/Z isomers, as well as mixtures of E/Z isomers.
7. Use of a compound according to count 5 or count 6 as precursor for the preparation of an ¹⁸F labelled PET imaging tracer.
8. Method for the preparation of a compound of formula III.

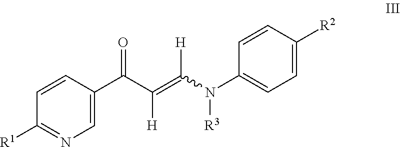

wherein
R¹ is selected from the group consisting of F, Cl, Br, I, NO₂, N⁺(CH₃)₃, I⁺aryl, and S⁺aryl_{2;}
R² is selected from the group consisting of H, OCH₃, and N(CH₃)₂; and
R³ is H;
   the method comprising the steps
providing a compound of formula IV;

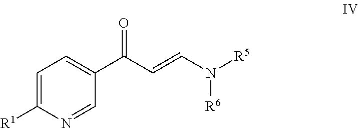

wherein
R¹ is selected from the group consisting of F, Cl, Br, I, NO₂, N⁺(CH₃)₃, I⁺aryl, and S⁺aryl₂;
R⁵, R⁶ independently are selected from the group consisting of H and alkyl;
providing a compound of formula V;

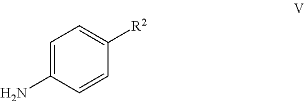

wherein R² is selected from the group consisting of H, OCH₃, and N(CH₃)₂;
   reacting the compound of formula IV with the compound of formula V to get the compound of formula III.
      9. Method for the preparation of a compound of formula IIa

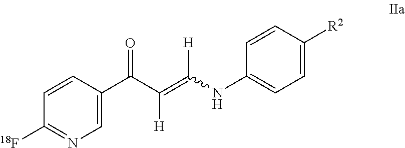

the method comprising the steps
providing a compound of formula III

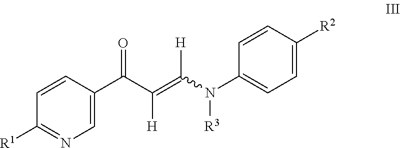

wherein
R¹ is selected from the group consisting of F, Cl, Br, I, NO₂, N⁺(CH₃)₃, I⁺aryl, and S⁺aryl_{2;}
R² is selected from the group consisting of H, OCH₃, and N(CH₃)₂; and
R³ is H or C(O)OC(CH₃)₃;
reacting the compound of formula III with a ¹⁸F-fluorination agent;
optionally, replacing the C(O)OC(CH₃)₃ group by an H atom, if R³ is C(O)OC(CH₃)₃.
   10. Method for imaging or diagnosing of diseases associated with amyloid deposits comprising the steps:
administering to a mammal an effective amount of a composition comprising a compound of formula I
obtaining PET images of the mammal and
assessing PET images.
   11. Composition comprising a compound according to counts 1, 2, 3, or 4 and at least one physiologically acceptable carrier, diluent, adjuvant or excipient.
   12. A kit comprising a sealed vial containing a predetermined quantity of a compound according to anyone of the counts 1 to 7, or stereoisomers thereof or their mixtures, or suitable salts of inorganic or organic bases or acids thereof, hydrates, complexes, esters, amides, or solvates thereof.

### General Synthesis of Compounds of the Invention

The compounds of the present invention as well as intermediates of said compounds can be prepared according to preparation methods well known to the person of ordinary skill in the art.

Schemes 1, 2a and 2b show potential pathways for preparing compounds and intermediates of the present invention. The reaction pathways are further described in the experimental section.

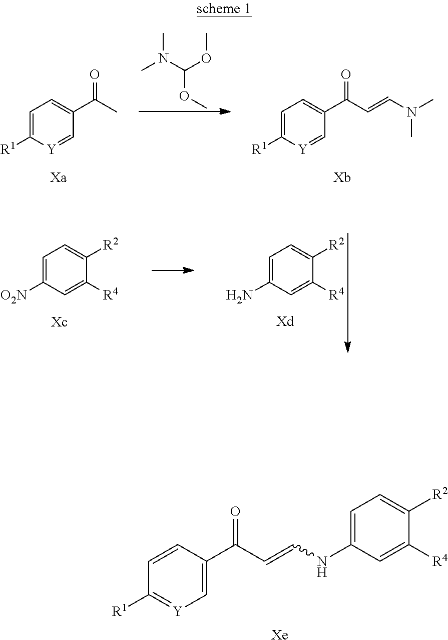

Compounds of the invention can be prepared e.g. by condensation of methyl aryl ketones Xa with N,N-dimethylformamide dimethylacetal to dimethylamino-1-aryl-propenones Xb. Enamine condensation of Xb with the aniline Xd derived from the nitroaryl compound Xc delivered the exemplified cold references 10-11, halogene- and nitro-precursors 15-18.

### [¹⁸F] Radiolabelling Procedures

[¹⁸F] radiolabeling procedures are well known to the person skilled in the art. [¹⁸F] radiolabeling can be preformed as shown in schemes 2a and 2b and as described below.

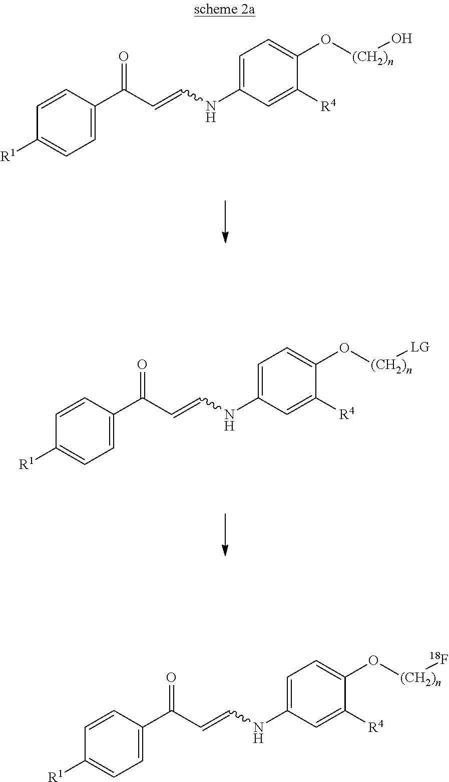

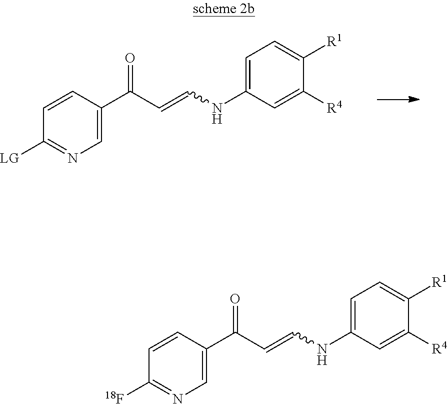

The leaving group LG e.g. can be selected from the group comprising halo, in particular chloro, bromo, iodo, nitro, mesyloxy, tosyloxy, trifluormethylsulfonyloxy, nonafluorobutylsulfonyloxy, (4-bromo-phenyl)sulfonyloxy, (4-nitro-phenyl)sulfonyloxy, (2-nitrophenyl)sulfonyloxy, (4-isopropyl-phenyl)sulfonyloxy, (2,4,6-tri-isopropyl-1-phenyl)sulfonyloxy, (2,4,6-trimethyl-phenyl)sulfonyloxy, (4-tertbutyl-phenyl)sulfonyloxy, and (4-methoxy-phenyl)sulfonyloxy.

[¹⁸F]Fluoride can be produced by proton bombardment in a cyclotron using a silver target (1 mL) filled with [¹⁸O]water for the ¹⁸O (p,n) ¹⁸F reaction. The aqueous [¹⁸F]fluoride can be passed through a cartridge (e.g. QMA-resin cartridge Waters, Sep Pak Light QMA Part. No.: WAT023525). The trapped [¹⁸F]fluoride can then be eluted from the cartridge by adding e.g. a Kryptofix K2.2.2/K₂CO₃ solution (Kryptofix is 4,7,13,16,21,24-Hexaoxa-1,10-diazabicyclo[8.8.8]-hexacosane). The nucleophilic substitution of the precursor works preferably in the presence of a base such as Tetrabutylammonium hydroxide (NBu₄OH), Tetrabutylammonium carbonate ((NBu₄)₂CO₃), Tetrabutylammonium hydrogencarbonate (NBu₄HCO₃), K₂CO₃ etc. and at elevated temperatures. The addition of crown ethers such as Kryptofix (K2.2.2) can influence the reaction positively, especially in the presence of K₂CO₃ as the base.

The potassium fluoride Kryptofix complex is preferably dried by repeated azeotropic distillation with sequential addition of acetonitrile. Solvents such as acetonitrile, DMF, DMSO etc. can be used as a reaction solvent. The labeling product can be purified by solid phase extraction using cartridges. Preferred cartridges are Sep-Pak Plus C18 cartridge (Waters, WAT020515). The cartridge can be rinsed with water and the compound can be eluted with acetonitrile. The eluted compound can be diluted with water and can then be subjected to preparative HPLC purification. Preferred HPLC columns are reversed phase columns such as Gemini 5μ C18 110 Å, 250*10 mm (Phenomenex, 00G-4435-N0). Mixtures of buffer solution, acids, water etc. with organic solvents such as acetonitrile, methanol, ethanol etc. can be used as mobile.

The solution can then be diluted with e.g. water to be passed through a cartridge for concentration and solvent change.

Preferably, the radio-labelling of the 2-halogene or 2-nitropyridyl precursors 15-18 is accomplished by treatment of the precursors 15-18 with ¹⁸F potassium fluoride and Kryptofix 222 in DMSO/MeCN to give the ¹⁸F labelled compound 18F-28.

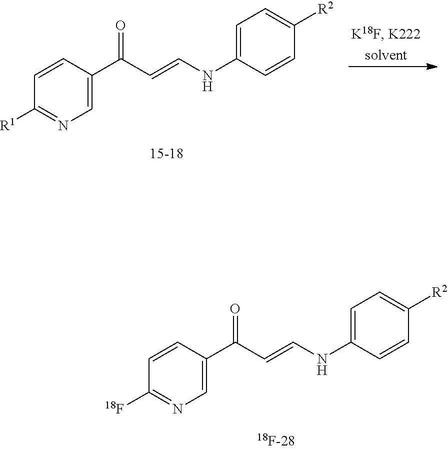

### BRIEF DESCRIPTION OF THE FIGURES

FIG. 1 Preparative HPLC chromatogram for purification of compound 28

FIG. 2 Analytical HPLC chromatogram of compound 28 (Gamma detection).

FIG. 3 Analytical HPLC chromatogram of compound 10 (UV detection).

FIG. 4 Autoradiographical analysis of binding of compound 28 to brain sections from cortex of Alzheimer's disease patients (AD) with amyloid beta plaques. Control sections without amyloid beta pathology stem from healthy volunteers (HC) and fronto-temporal dementia (FTD) patients. Blocking of specific signals was performed with an excess of cold compound. Arrows point to plaque-specific signals.

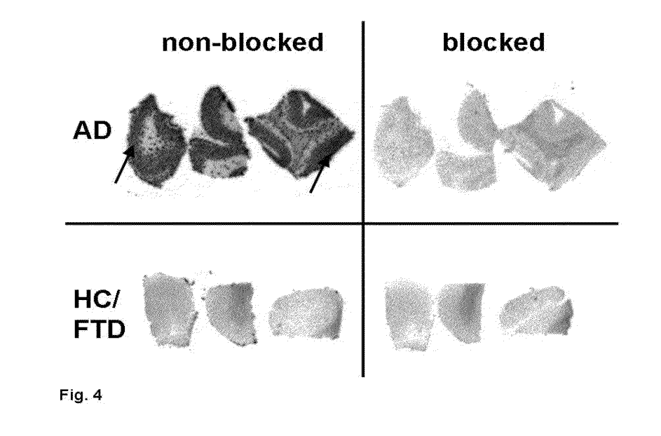

### EXPERIMENTAL SECTION

General: All solvents and chemicals were obtained from commercial sources and used without further purification if not stated otherwise. The following table lists the abbreviations used in this paragraph and in the Examples section as far as they are not explained within the text body.

Abbreviations

| | |
|---|---|
| Bq | Bequerel |
| br | broad signal (in NMR) |
| d | doublet |
| dd | doublet of doublet |
| ddd | doublet of doublet of doublet |
| GBq | giga Bequerel |
| h | hour |
| HPLC | High Performance Liquid Chromatography |
| K_{2.2.2} | Kryptofix 2.2.2 |
| kBq | kilo Bequerel |
| m | multiplet |
| MBq | mega Bequerel |
| min | minute |
| PBS | phosphate buffered saline |
| r.t. | room temperature |
| s | singlet |
| sec | second |
| t | triplet |

### Preparation of Intermediates

### General Procedure for N,N-Dimethylformamide Dimethyl Acetal Condensation (B)

The acetophenone (25 mmol) was solved in ethanol (20 mL) and N,N-dimethylformamide dimethyl acetal (37 mmol) was added. The reaction mixture was stirred under reflux for 6 h and concentrated. The residue was elutriated in hexane and the remaining precipitate was filtered off, washed several times with hexane and dried.

### Intermediate B1: (E)-3-(Dimethylamino)-1-(4-fluorophenyl)prop-2-en-1-one

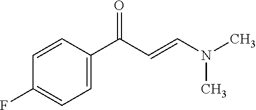

Yield: 76%

¹H-NMR (300 MHz, d6-DMSO): α=7.92 (m, 2H), 7.67 (d, 1H), 7.20 (m, 2H), 5.77 (d, 1H), 3.10 (bs, 3H), 2.87 (br s, 3H) ppm.

### Intermediate B2: (E)-3-(Dimethylamino)-1-[4-fluoro-3-(trifluoromethyl)phenyl]prop-2-en-1-one

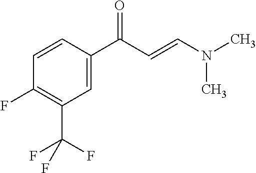

Yield: 54%

¹H-NMR (300 MHz, d6-DMSO): δ=8.26 (m, 1H), 8.16 (m, 1H), 7.75 (d, 1H), 7.54 (m, 1H), 5.87 (d, 1H), 3.13 (bs, 3H), 2.92 (br s, 3H) ppm.

ESI-MS m/z 262 (M+1).

### Intermediate B3: 5-[(E)-3-(Dimethylamino)acryloyl)-2-fluorobenzonitrile

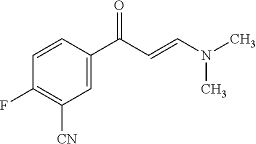

Yield: 20%

¹H-NMR (300 MHz, d6-DMSO): δ=8.46 (dd, 1H), 8.25 (m, 1H), 7.74 (d, 1H), 7.56 (t, 1H), 5.89 (d, 1H), 3.10 (d, 3H), 2.90 (d, 3H) ppm.

### Intermediate B4: (E)-3-(Dimethylamino)-1-(6-fluoropyridin-3-yl)prop-2-en-1-one

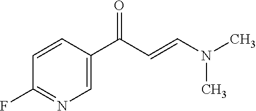

Yield: 85%

¹H-NMR (400 MHz, d6-DMSO): δ=8.73 (d, 1H), 8.38 (ddd, 1H), 7.73 (d, 1H), 7.20 (dd, 1H), 5.82 (d, 1H), 3.13 (bs, 3H), 2.90 (br s, 3H) ppm.

ESI-MS m/z 195 (M+1).

### Intermediate B5: (E)-1-Biphenyl-4-yl-3-(dimethylamino)prop-2-en-1-one

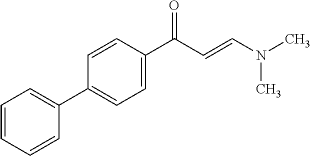

Yield: 79%

¹H-NMR (300 MHz, CDCl₃): δ=8.03 (d, 2H), 7.89 (d, 1H), 7.67-7.70 (m, 4H), 7.48-7.53 (m, 2H), 7.41 (m, 1H), 5.82 (d, 1H), 3.19 (bs, 3H), 3.01 (br s, 3H) ppm.

ESI-MS m/z 252 (M+1).

### Intermediate B6: (E)-1-(6-Bromopyridin-3-yl)-3-(dimethylamino)prop-2-en-1-one

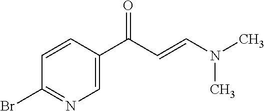

Yield: 55%

¹H-NMR (400 MHz, d6-DMSO): δ=8.80 (d, 1H), 8.10 (dd, 1H), 7.70 (d, 1H), 7.65 (d, 1H), 5.80 (d, 1H), 3.15 (s, 3H), 2.90 (s, 3H) ppm.

### Intermediate B7: (E)-1-(6-Chloropyridin-3-yl)-3-(dimethylamino)prop-2-en-1-one

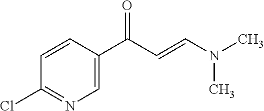

Yield: 52%

¹H-NMR (400 MHz, d6-DMSO): δ=8.89 (d, 1H), 8.23 (dd, 1H), 7.73 (d, 1H), 7.52 (d, 1H), 5.81 (d, 1H), 3.13 (s, 3H), 2.91 (s, 3H) ppm.

### Intermediate B8: (E)-3-(Dimethylamino)-1-(6-nitropyridin-3-yl)prop-2-en-1-one

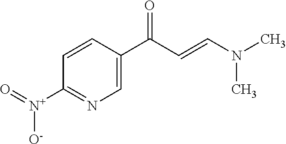

Yield: 45%

¹H-NMR (400 MHz, d6-DMSO): δ=9.06 (s, 1H), 8.57 (dd, 1H), 8.31 (d, 1H), 7.80 (d, 1H), 5.90 (d, 1H), 3.19 (s, 3H), 2.96 (s, 3H) ppm.

### Intermediate B9: (E)-3-(Dimethylamino)-1-(6-iodopyridin-3-yl)prop-2-en-1-one

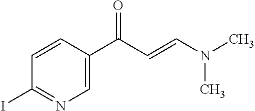

Yield: 63%

¹H-NMR (300 MHz, CDCl₃): δ=8.80 (s, 1H), 7.75-7.95 (ser m, 3H), 5.58 (d, 1H), 3.19 (s, 3H), 2.93 (s, 3H) ppm.

### General Procedure Nitrophenol Alkylation (C)

Nitrophenol (18 mmol) was solved in N,N-dimethylformamide (16 mL) followed by the addition of caesium carbonate (18 mmol) and the alkylbromide (27 mmol). The reaction mixture was heated at 100° C. for 15 min under microwave irradiation and was then concentrated. The residue was taken up in water and dichloromethane. The phases were separated and the aqueous phase was extracted three times with dichloromethane. The combined organic phases were dried over sodium sulfate, filtrated and concentrated. The residue was purified by chromatography on silica gel.

### Intermediate C1: 1-(2-Fluoromethoxy)-4-nitrobenzene

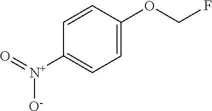

Yield: 84%

¹H-NMR (400 MHz, CDCl₃): δ=8.25 (d, 2H), 7.17 (d, 2H), 5.79 (d, 2H) ppm.

CI+−MS m/z 189 (M+1+NH₃).

### Intermediate C2: 1-(2-Fluoroethoxy)-4-nitrobenzene

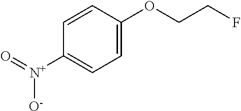

Yield: 82%

¹H-NMR (300 MHz, CDCl₃): δ=8.21 (d, 2H), 6.99 (d, 2H), 4.78 (dm, 2H), 4.33 (dm, 2H) ppm. CI+−MS m/z 203 (M+1+NH₃).

### Intermediate C3: 1-(3-Fluoropropoxy)-4-nitrobenzene

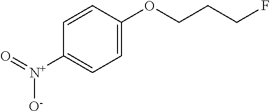

Yield: 68%

¹H-NMR (300 MHz, CDCl₃): δ=8.21 (d, 2H), 6.97 (d, 2H), 4.66 (td, 2H), 4.20 (t, 2H), 2.23 (dp, 2H) ppm.

### Intermediate C4: 2-(4-N itrophenoxy)ethyl acetate

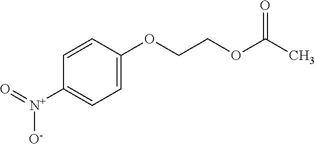

Yield: 58%

¹H-NMR (300 MHz, CDCl₃): δ=8.21 (d, 2H), 6.97 (d, 2H), 4.46 (t, 2H), 4.27 (t, 2H), 2.11 (s, 3H) ppm.

CI+−MS m/z 243 (M+1+NH₃).

### Intermediate C5: 3-(4-Nitrophenoxy)propan-1-ol

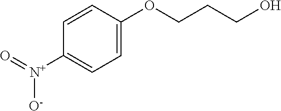

Yield: 75%

¹H-NMR (300 MHz, CDCl₃): δ=8.25 (1/2 ABq, 2H), 6.98 (1/2ABq, 2H), 4.26 (m, 2H), 3.89 (m, 2H), 2.08 (m, 2H) ppm.

### General Procedure for Nitro Group Reduction (D)

Nitrobenzene C (5.84 mmol) was solved in ethanol (40 mL) followed by the addition of Pd/C (0.94 mmol). The mixture was stirred under hydrogen over night, filtrated and concentrated. The residue was purified by chromatography on silica gel.

### Intermediate D1: 4-(Fluoromethoxy)aniline

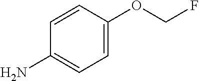

Yield: 98%

¹H-NMR (300 MHz, CDCl₃): δ=6.91 (d, 2H), 6.64 (d, 2H), 5.61 (d, 2H), 3.56 (bs, 2H) ppm.

### Intermediate D2: 4-(2-Fluoroethoxy)aniline

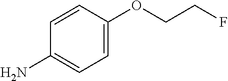

Yield: 87%

¹H-NMR (400 MHz, CDCl₃): δ=6.78 (d, 2H), 6.64 (d, 2H), 4.70 (dm, 2H), 4.14 (dm, 2H), 3.44 (bs, 2H) ppm.

CI+−MS m/z 173 (M+1+NH₃).

### Intermediate D3: 4-(3-Fluoropropoxy)aniline

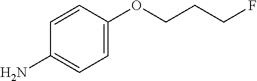

Yield: 54%

¹H-NMR (300 MHz, CDCl₃): δ=6.75 (d, 2H), 6.64 (d, 2H), 4.64 (dt, 2H), 4.02 (t, 2H), 3.25 (bs, 2H), 2.13 (dp, 2H) ppm.

### Intermediate D4: 2-(4-Aminophenoxy)ethyl acetate

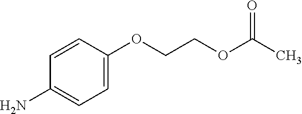

Yield: 88%

¹H-NMR (300 MHz, CDCl₃): δ=6.81 (d, 2H), 6.68 (d, 2H), 4.42 (t, 2H), 4.14 (t, 2H), 3.30 (bs, 2H), 2.14 (s, 3H) ppm.

CI+−MS m/z 213 (M+1+NH₃).

### Intermediate D5: 3-(4-Aminophenoxy)propan-1-ol

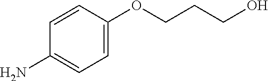

Yield: 77%

¹H-NMR (300 MHz, d6-DMSO): δ=6.58 (1/2 ABq, 2H), 6.46 (1/2ABq, 2H), 4.58 (br s, 2H), 4.48 (m, 1H), 3.81 (m, 2H), 3.50 (m, 2H), 1.75 (m, 2H) ppm.

### Intermediate D6: 3-(4-Aminophenyl)propan-1-ol

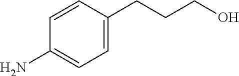

Yield: 99%

¹H-NMR (400 MHz, CDCl₃): δ=6.99 (d, 2H), 6.63 (d, 2H), 3.66 (t, 2H), 2.60 (t, 2H), 1.84 (p, 2H) ppm.

CI+−MS m/z 213 (M+1+NH₃).

### Preparation of Compounds According to the Invention

### General Procedure for Enamine Condensation

(Dimethylamino)prop-2-en-1-one (1 mmol) and the aniline (1 mmol) were solved in acetic acid (3 mL) and stirred at 90° C. for 10 min. After cooling to rt the precipitate was filtered off and washed with ethyl acetate and ether and dried.

### Example 1

### (E/Z)-3-{[4-(Fluoromethoxy)phenyl]amino}-1-phenylprop-2-en-1-one

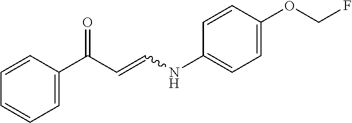

Yield: 47%

¹H-NMR (300 MHz, d6-DMSO): E/Z 0.6/1 δ=12.04 (d, 1H), 10.06 (bd, 0.6H), 8.00 (dd, 0.6H), 7.90 (d, 2H), 7.82 (m, 2.5H), 7.51-7.45 (m, 5H), 7.31 (d, 2H), 7.15-7.04 (m, 4.5H), 6.36 (d, 0.6H), 6.08 (d, 1H), 5.79 (d, 2H), 5.77 (d, 1.2H) ppm.

### Example 2

### (E/Z)-3-{[4-(2-Fluoroethoxy)phenyl]amino}-1-phenylprop-2-en-1-one

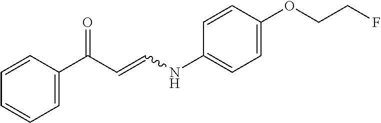

Yield: 72%

¹H-NMR (300 MHz, d6-DMSO): E/Z1/2 δ=12.08 (d, 1H), 10.0 (bs, 0.5H), 8.00 (d, 0.5H), 7.91 (d, 2H), 7.82 (m, 2.5H), 7.53-7.43 (m, 5H), 7.26 (d, 1H), 7.08 (d, 1H), 6.93 (m, 3H), 6.32 (d, 0.5H), 6.03 (d, 1H), 4.70 (dm, 3H), 4.17 (dm, 3H) ppm.

ESI-MS m/z 286.4 (M+1).

### Example 3

### (E/Z)-1-(4-Bromophenyl)-3-{[4-(2-fluoroethoxy)phenyl]amino}prop-2-en-1-one

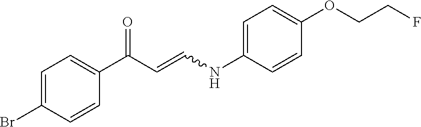

Yield: 64%

¹H-NMR (300 MHz, d6-DMSO): E/Z 0.6/1 δ=12.08 (d, 1H), 10.09 (bs, 0.5H), 8.04 (bd, 0.6H), 7.85 (d, 2H), 7.82 (d, 1.2H), 7.75 (d, 1.2H), 7.65 (d, 2H), 7.64 (d, 1.2H), 7.27 (d, 2H), 7.10 (d, 1.2H), 6.97-6.93 (m, 3.2H), 6.28 (d, 0.6H), 6.02 (d, 1H), 4.70 (dm, 3.2H), 4.19 (dm, 3.2H) ppm.

ESI-MS m/z 366 (M+1).

### Example 4

### (Z)-3-{[4-(3-Fluoropropoxy)phenyl]amino}-1-phenylprop-2-en-1-one

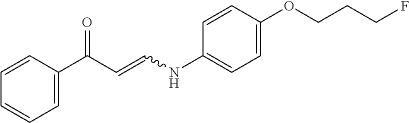

Yield: 27%

¹H-NMR (300 MHz, CDCl₃): only signals of Z-isomer detected: 12.19 (d, 1H), 7.93 (d, 2H), 7.52-7.43 (m, 4H), 7.05 (d, 2H), 6.90 (d, 2H), 5.98 (d, 1H), 4.65 (dt, 2H), 4.09 (t, 2H), 2.17 (dp, 2H) ppm.

### Example 5

### (E/Z)-1-(4-Fluorophenyl)-3-[(4-methoxyphenyl)amino]prop-2-en-1-one

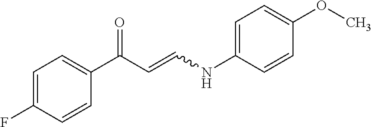

Yield: 57%

¹H-NMR (400 MHz, d6-DMSO): E/Z 0.6/1 δ=12.05 (d, 1H), 10.09 (bs, 0.5H), 7.98 (m, 2.6H), 7.88 (m, 1.2H), 7.81 (dd, 1H), 7.29-7.24 (m, 5.2H), 7.08 (d, 1.2H), 6.92-6.88 (m, 3.2H), 6.29 (d, 0.6H), 6.01 (d, 1H), 3.71 (s, 3H), 3.69 (s, 1.8H) ppm.

ESI-MS m/z 272 (M+1).

### Example 6

### (E/Z)-3-{[4-(Dimethylamino)phenyl]amino}-1-(4-fluorophenyl)prop-2-en-1-one

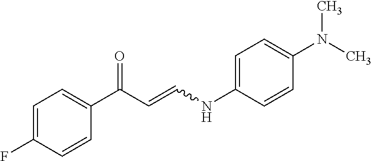

Yield: 44%

¹H-NMR (300 MHz, d6-DMSO): E/Z 0.5/1 δ=12.14 (d, 1H), 10.09 (bs, 0.4H), 7.96 (m, 2.5H), 7.85 (m, 1H), 7.77 (dd, 1H), 7.28-7.24 (m, 3H), 7.15 (d, 2H), 7.01 (d, 1H), 6.71 (d, 3H), 6.24 (d, 0.5H), 5.96 (d, 1H), 2.84 (s, 6H), 2.82 (s, 3H) ppm.

ESI-MS m/z 285 (M+1).

### Example 7

### (E/Z)-3-{[4-(Dimethylamino)phenyl]amino}-1-[4-fluoro-3-(trifluoromethyl)phenyl]prop-2-en-1-one

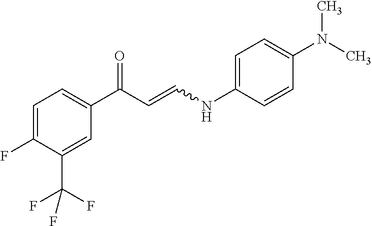

Yield: 48%

¹H-NMR (300 MHz, d6-DMSO): E/Z 0.6/1 δ=12.23 (d, 1H), 10.18 (bs, 0.4H), 8.28 (m, 1H), 8.21-8.08 (m, 2.6H), 7.85 (dd, 1H), 7.58 (dd, 1.6H), 7.19 (d, 2H), 7.02 (d, 1.2H), 6.74-6.69 (m, 3.2H), 6.26 (d, 0.6H), 6.06 (d, 1H), 2.85 (s, 6H), 2.82 (s, 3.6H) ppm.

ESI-MS m/z 353 (M+1).

### Example 8

### 5-[(E/Z)-3-{[4-(Dimethylamino)phenyl]amino}acryloyl]-2-fluorobenzonitrile

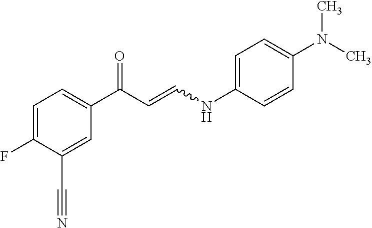

Yield: 10%

¹H-NMR (300 MHz, d6-DMSO): δ=12.20 (d, 1H), 8.43 (dd, 1H), 8.27 (m, 1H), 7.88 (dd, 1H), 7.60 (t, 1H), 7.20 (d, 2H), 6.72 (d, 2H), 6.06 (d, 1H), 2.85 (s, 6H) ppm.

### Example 9

### (E/Z)-1-[4-Fluoro-3-(trifluoromethyl)phenyl]-3-[(4-methoxyphenyl)amino]prop-2-en-1-one

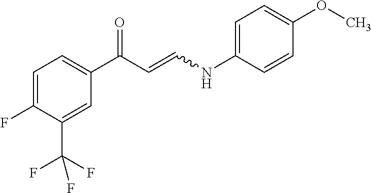

Yield: 25%

¹H-NMR (300 MHz, d6-DMSO): E/Z 0.6/1 δ=12.10 (d, 1H), 10.19 (bs, 0.5H), 8.30 (m, 1H), 8.22-8.08 (m, 3.0H), 7.87 (dd, 1H), 7.63 (dd, 1.2H), 7.29 (d, 2H), 7.11 (d, 1.2H), 6.95-6.88 (m, 3.4H), 6.31 (d, 0.6H), 6.10 (d, 1H), 3.71 (s, 3H), 3.70 (s, 1.8H) ppm.

ESI-MS m/z 340 (M+1).

### Example 10

### (E/Z)-3-{[4-(Dimethylamino)phenyl]amino}-1-(6-fluoropyridin-3-yl)prop-2-en-1-one

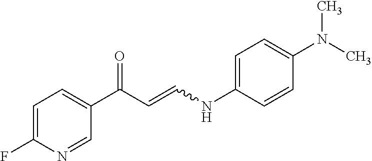

Yield: 58%

¹H-NMR (400 MHz, d6-DMSO): E/Z 0.5/1 δ=12.21 (d, 1H), 10.2 (bs, 0.4H), 8.77 (d, 1H), 8.64 (d, 0.5H), 8.42 (ddd, 1H), 8.32 (ddd, 0.5H), 7.84 (dd, 1H), 7.27-7.18 (m, 3.5H), 7.12-7.02 (m, 1.5H), 6.73-6.68 (m, 3.5H), 6.01 (d, 1H), 2.84 (s, 9H), 2.82 (s, 3H) ppm.

ESI-MS m/z 286 (M+1).

### Example 11

### (E/Z)-1-(6-Fluoropyridin-3-yl)-3-[(4-methoxyphenyl)amino]prop-2-en-1-one

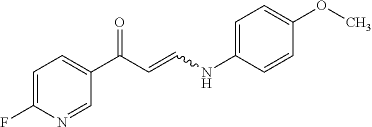

Yield: 39%

¹H-NMR (400 MHz, d6-DMSO): E/Z 0.6/1 δ=12.19 (d, 1H), 10.22 (bs, 0.4H), 8.79 (d, 1H), 8.66 (d, 0.6H), 8.45 (ddd, 1H), 8.33 (ddd, 0.6H), 8.09 (bm, 0.6H), 7.87 (dd, 1H), 7.30-7.20 (m, 3.6H), 7.13 (d, 1.2H), 6.93-6.88 (m, 3.2H), 6.27 (d, 0.6H), 6.06 (d, 1H), 3.71 (s, 3H), 3.69 (s, 1.8H) ppm.

ESI-MS m/z 273 (M+1).

### Example 12

### (E/Z)-3-[(4-Fluorophenyl)amino]-1-phenylprop-2-en-1-one

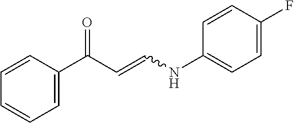

Yield: 81%

¹H-NMR (300 MHz, d6-DMSO): E/Z 0.65/1 δ=12.04 (d, 1H), 10.15 (bs, 0.6H), 8.08 (m, 0.65H), 7.97 (dd, 2H), 7.90-7.84 (m, 2.2H), 7.59-7.45 (m, 5H), 7.43-7.37 (m, 2H), 7.25-7.15 (m, 4.7H), 6.42 (d, 0.65H), 6.13 (d, 1H) ppm.

ESI-MS m/z 242 (M+1).

### Example 13

### (E/Z)-3-[(3-Bromo-4-fluorophenyl)amino]-1-phenylprop-2-en-1-one

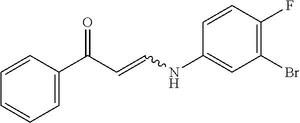

Yield: 85%

¹H-NMR (300 MHz, d6-DMSO): E/Z 0.65/1 δ=11.89 (d, 1H), 10.12 (bs, 0.6H), 8.04 (m, 0.65H), 7.97 (dd, 2H), 7.87-7.76 (m, 3H), 7.56-7.44 (m, 6H), 7.38-7.26 (m, 2.6H), 7.19-7.15 (m, 0.65H), 6.40 (d, 0.65H), 6.11 (d, 1H) ppm.

ESI-MS m/z 322 (M+1).

### Example 14

### (E/Z)-1-Biphenyl-4-yl-3-{[4-(2-fluoroethoxy)phenyl]amino}prop-2-en-1-one

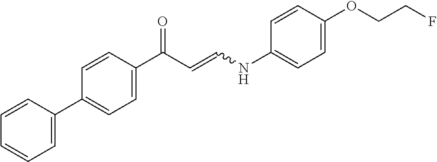

Yield: 83%

¹H-NMR (400 MHz, d6-DMSO): E/Z 0.6/1 δ=12.11 (d, 1H), 10.03 (bs, 0.5H), 8.01 (d, 2H), 7.91 (d, 1.2H), 7.84 (dd, 1.2H), 7.76 (d, 3.2H), 7.71 (dm, 3.2H), 7.47 (t, 3.2H), 7.38 (t, 1.6H), 7.28 (d, 2H), 7.10 (d, 1.2H), 6.95 (t, 3.2H), 6.38 (d, 0.6H), 6.09 (d, 1H), 4.70 (dm, 3.2H), 4.19 (dm, 3.2H) ppm. ESI-MS m/z 362 (M+1).

### Example 15

### (E/Z)-1-(6-Bromopyridin-3-yl)-3-{[4-(dimethylamino)phenyl]amino}prop-2-en-1-one

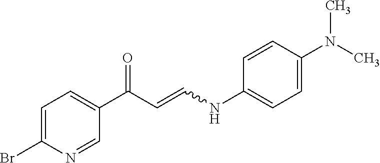

Yield: 77%

¹H-NMR (400 MHz, CDCl₃): δ=12.32 (d, 1H), 8.80 (d, 1H), 8.05 (d, 1H), 7.57 (d, 1H), 7.50 (m, 1H), 7.07 (d, 2H), 6.72 (d, 2H), 5.88 (d, 1H), 3.00 (s, 6H) ppm.

### Example 16

### (E/Z)-1-(6-Chloropyridin-3-yl)-3-{[4-(dimethylamino)phenyl]amino}prop-2-en-1-one

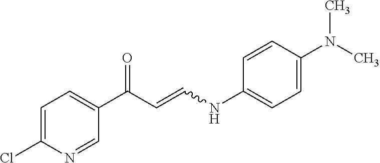

Yield: 65%

¹H-NMR (400 MHz, CDCl₃): δ=12.30 (d, 1H), 8.90 (d, 1H), 8.19 (d, 1H), 7.48-7.54 (m, 1H), 7.40 (d, 1H), 7.06 (d, 2H), 6.73 (d, 2H), 5.88 (d, 1H), 2.96 (s, 6H) ppm.

### Example 17

### (E/Z)-3-{[4-(Dimethylamino)phenyl]amino}-1-(6-nitropyridin-3-yl)prop-2-en-1-one

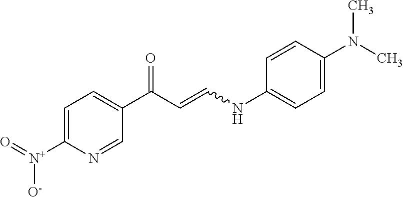

Yield: 92%

¹H-NMR (400 MHz, CDCl₃): δ=12.45 (br s, 1H), 9.09 s, 1H), 8.50 (d, 1H), 8.32 (d, 1H), 7.58 (m, 1H), 7.08 (d, 2H), 6.77 (br s, 2H), 5.91 (d, 1H), 3.00 (s, 6H) ppm.

### Example 18

### (E/Z)-3-{[4-(Dimethylamino)phenyl]amino}-1-(6-iodopyridin-3-yl)prop-2-en-1-one

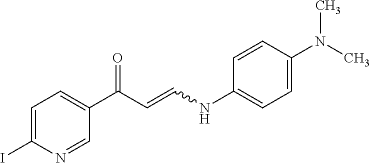

Yield: 65%

¹H-NMR (300 MHz, CDCl₃): δ=12.30 (d, 1H), 8.82 (s, 1H), 7.80 (s, 2H), 7.45 (dd, 1H), 7.08 (1/2 ABq, 2H), 6.75 (br s, 2H), 5.86 (d, 1H), 2.98 (s, 6H) ppm.

### Example 19

### (E/Z)-3-{[4-(3-Fluoropropyl)phenyl]amino}-1-phenylprop-2-en-1-one

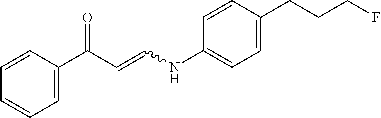

Yield: 30%

¹H-NMR (400 MHz, CDCl₃): δ=12.15 (d, 1H), 7.93 (d, 2H), 7.40-7.56 (m, 4H), 7.19 (d, 2H), 7.08 (d, 2H), 6.01 (d, 1H), 4.40-4.55 (m, 2H), 2.72 (m, 2H), 1.90-2.10 (m, 2H) ppm.

### Example 20

### (E/Z)-3-{[4-(2-Hydroxyethoxy)phenyl]amino}-1-phenylprop-2-en-1-one

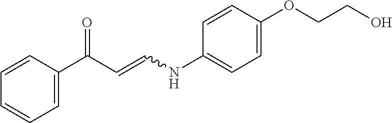

Yield: 45%

¹H-NMR (300 MHz, d6-DMSO) δ=12.09 (d, 1H), 7.90 (m, 2H), 7.77 (m, 2H), 7.45 (m, 1H), 7.23 (d, 2H), 7.08 (d, 1H), 6.90 (d, 2H), 6.02 (d, 1H), 4.84 (m, 1H), 3.92 (m, 2H), 3.66 (m, 2H) ppm.

### Example 21

### 3-[4-(3-Hydroxypropoxy)phenylamino]-1-phenylpropenone

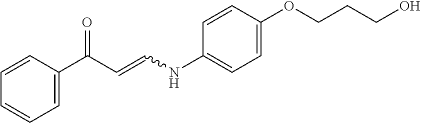

Yield: 62%

¹H-NMR (300 MHz, CDCl₃): δ=12.20 (d, 1H), 7.95 (d, 2H), 7.40-7.30 (m, 4H), 7.05 (d, 2H), 6.90 (d, 2H), 6.01 (d, 1H), 4.01 (m, 2H), 3.89 (m, 2H), 2.05 (m, 2H) ppm.

### Example 22

### (E/Z)-3-{[4-(3-Hydroxypropyl)phenyl]amino}-1-phenylprop-2-en-1-one

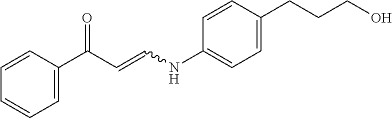

Yield: 56%

¹H-NMR (300 MHz, CDCl₃): Z only: δ=12.13 (d, 1H), 7.93 (d, 2H), 7.54-7.43 (m, 4H), 7.17 (d, 2H), 7.04 (d, 2H), 6.01 (d, 1H), 3.69 (t, 2H), 2.69 (t, 2H), 1.88 (p, 2H) ppm.

### General Procedure for Sulfonyl Precursor Formation

Alcohol 20 (0.35 mmol) was solved in dichloromethane (7 mL) at 0° C. followed by the addition of triethylamine (1 mmol) and sulfonic acid chloride (0.53 mmol). The mixture was stirred at r.t. for 1 h, diluted with dichloromethane, washed with saturated ammonium chloride solution, dried over sodium sulfate, filtrated and concentrated. The residue was purified by chromatography on silica gel to give compound 23.

### Example 23

### 2-({4-[(E/Z)-(3-Oxo-3-phenylprop-1-en-1-yl)amino]phenyl}oxy)ethyl methane-sulfonate

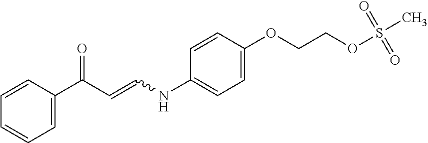

Yield: 70%

¹H-NMR (300 MHz, CDCl₃): only signals of Z-isomer detected: δ=12.18 (d, 1H), 7.93 (d, 2H), 7.53-7.40 (m, 4H), 7.06 (d, 2H), 6.90 (d, 2H), 6.00 (d, 1H), 4.57 (t, 2H), 4.23 (t, 2H), 3.10 (s, 3H) ppm.

ESI-MS m/z 362 (M+1).

### Example 24

### 3-({4-[(E/Z)-(3-Oxo-3-phenylprop-1-en-1-yl)amino]phenyl}oxy)propyl methanesulfonate

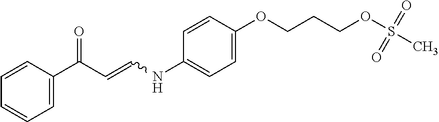

Yield: 71%

¹H-NMR (300 MHz, CDCl₃): δ=12.2 (d, 1H), 7.94 (d, 2H), 7.46 (m, 4H), 7.07 (d, 2H), 6.88 (d, 2H), 5.98 (d, 1H), 4.45 (m, 2H), 4.08 (m, 2H), 3.00 (s, 3H), 2.23 (m, 2H) ppm.

### Example 25

### 3-{4-[(E/Z)-(3-Oxo-3-phenylprop-1-en-1-vl)amino]phenyl}propyl methanesulfonate

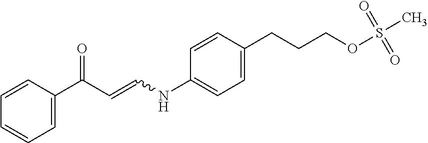

Yield: 60%

¹H-NMR (300 MHz, CDCl₃): δ=12.15 (d, 1H), 7.94 (d, 2H), 7.41-7.57 (m, 4H), 7.19 (d, 2H), 7.06 (d, 2H), 6.01 (d, 1H), 4.25 (m, 2H), 3.01 (s, 3H), 2.72 (m, 2H), 2.02-2.12 (m, 2H) ppm.

### General Procedure for BOC-Protection of Amino Propenones

The aminopropenone 17 (1 mmol) was dissolved in 10 mL of dry N,N-dimethylformamide containing triethylamine (1 equivalent) and di-tert-butyl dicarbonate (1.5 equivalents). The resulting solution was heated under an atmosphere of nitrogen and exclusion of moisture for 3 h at 80° C. Subsequently, the cooled reaction mixture was diluted with ethyl acetate, the organic phase was thoroughly washed with water and brine. After drying over sodium sulfate, the solution was filtered, the volatiles evaporated, and the product isolated by silica gel column chromatography.

### Example 26

### tert-Butyl N-[4-(dimethylamino)phenyl]-N-[(E/Z)-3-(6-nitropyridin-3-yl)-3-oxoprop-1-en-1-yl]carbamate

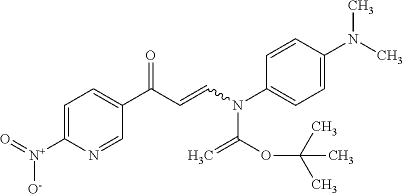

Yield: 30%

¹H-NMR (300 MHz, CDCl₃): δ=8.90 (s, 1H), 8.70 (d, 1H), 8.35 (d, 1H), 8.24 (d, 1H), 7.02 (d, 2H), 6.82 (d, 2H), 5.75 (d, 1H), 3.05 (s, 6H), 1.50 (s, 9H) ppm.

### Example 27

### tert-Butyl N-[4-(dimethylamino)phenyl]-N-[(E/Z)-3-(6-iodopyridin-3-yl)-3-oxoprop-1-en-1-yl]carbamate

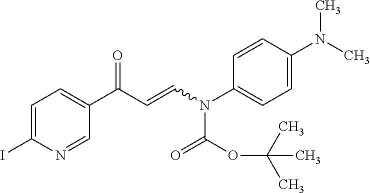

Yield: 10%

¹H-NMR (300 MHz, CDCl₃): δ=8.68 (d, 1H), 8.59 (s, 1H), 7.78 (d, 1H), 7.70 (d, 1H), 7.05 (s, 2H), 6.78 (d, 2H), 5.72 (d, 1H), 3.03 (s, 6H), 1.48 (s, 9H) ppm.

### Radiolabeling Examples

### Example 28

### (E/Z)-3-{[4-(Dimethylamino)phenyl]amino}-1-(6-[¹⁸F]-fluoropyridin-3-yl)prop-2-en-1-one

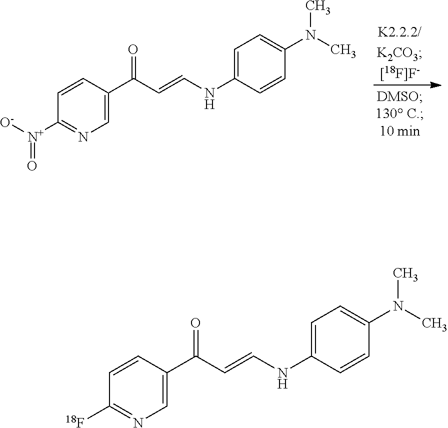

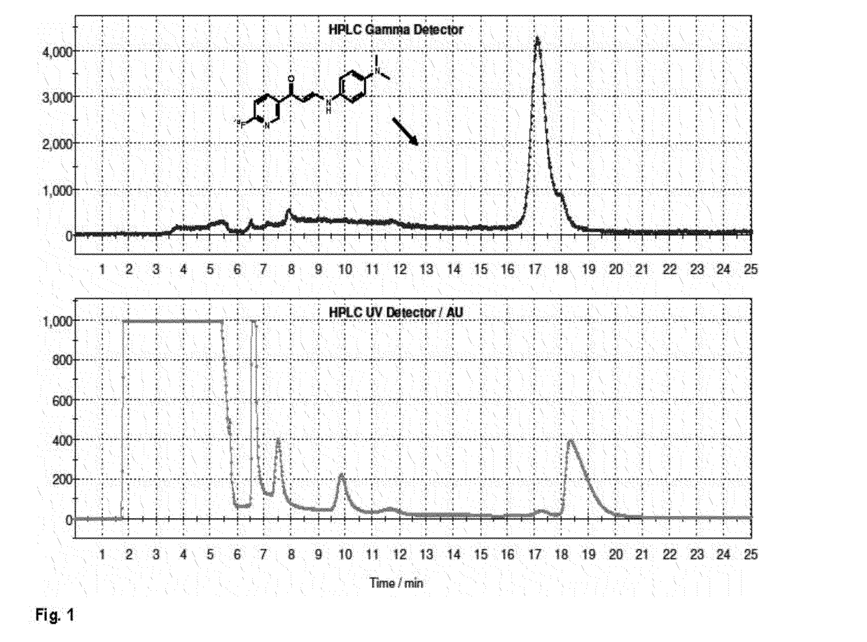

Aqueous [¹⁸F]Fluoride (14.4 GBq) was trapped on a QMA cartridge (Waters) and eluted with 2 mL Kryptofix-solution (5 mg potassium carbonate in 0.95 mL acetonitrile+1 mg K_{2.2.2} in 0.05 mL water) into the reactor. The solvent was removed by heating at 80° C. for 3 min (N₂ stream and vacuum) and at 120° C. for additional 3 min (vacuum). Anhydrous acetonitrile (1 mL) was added and evaporated as before. A solution of precursor 17 (5 mg) in 1 mL anhydrous dimethyl sulfoxide was added. After heating at 130° C. for 10 min the crude reaction mixture was cooled down to 50° C. and diluted with 10 mL water and pushed through a Sep-Pak C18 Plus cartridge (Waters). The F-18 labeled crude product was eluted from the cartridge with 2 mL acetonitrile and diluted with a 3 mL solvent-mixture (acetonitrile/water=18/82+0.1% trifluoro acetic acid) and purified by preparative HPLC: SEPSERV UltraSep ES AMID H RP18P 250×8 mm; 5 μm Ser. Nr. H01801; B. 730/07; isocratic, acetonitrile/water=18/82+0.1% trifluoro acetic acid, flow: 3 mL/min; t_{R}˜17 min (FIG. 1).

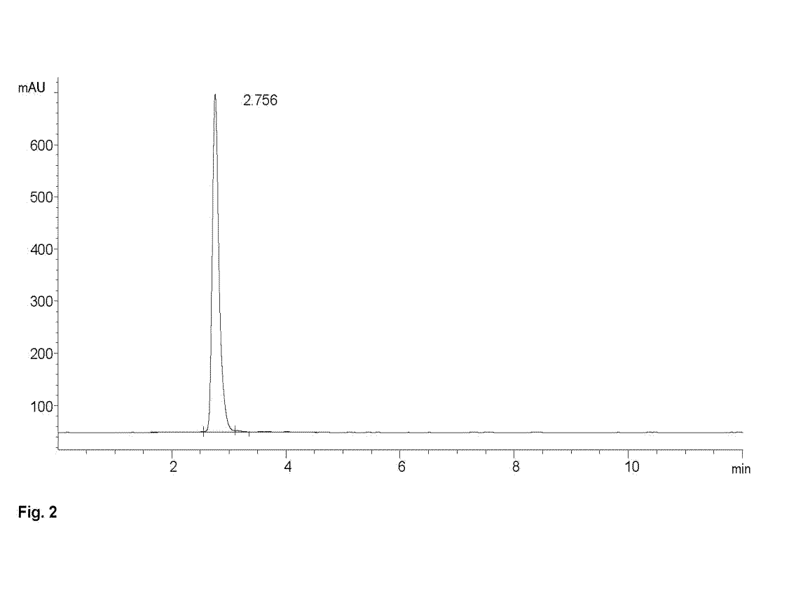

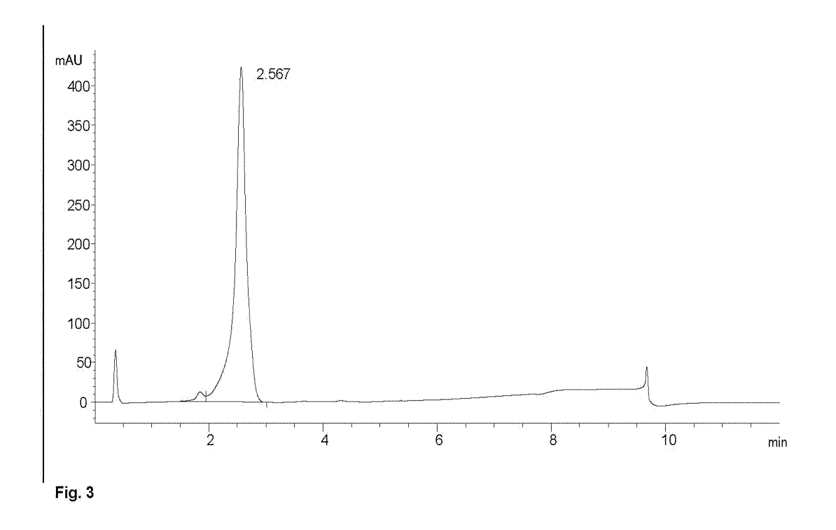

The collected HPLC fraction was diluted with 40 mL water and immobilized on a Sep-Pak plus short tC18 cartridge (Waters), which was washed with 10 mL water and eluted with 1 mL ethanol into the product vial to deliver the F-18 labeled product 28 (750 MBq) in a radiochemical yield of 8% corrected for decay (radiochemical purity >97% (HPLC). The desired F-18 labeled product 28 (FIG. 2, t_{R}=2.8 min) was analyzed using analytical HPLC: ACE3-C18 50 mm×4.6 mm; solvent gradient: start 5% acetonitrile-95% acetonitrile in 0.1% trifluoroacetic acid in 7 min., flow: 2 mL/min and confirmed by co-injection with the corresponding non-radioactive F-19 fluoro-standard 10 on the analytical HPLC (FIG. 3, t_{R}=2.6 min).

### Biological Methods and Results

### Binding Studies Using Human Brain Homogenate

A competition assay with a tritiated amyloid ligand was performed in 96-well plates (Greiner bio-one; Cat. 651201; Lot. 06260130) using brain homogenate from AD patients. Homogenates were prepared by homogenizing (Ultra-Turrax, setting 2, 30 s, 24000 rpm) dissected frontal cortex containing grey matter and white matter from AD patients in phosphate buffered saline (PBS, pH 7.4). The homogenate with a concentration of 100 mg wet tissue/mL was divided into aliquots of 300 μL and stored at −80° C. Varying concentrations of the unlabeled test substances were incubated with 100 μg/mL homogenate and 10 nM of the tritiated ligand in PBS, 0.1% BSA (final volume 200 μL) for 3 h at room temperature. Subsequently the binding mixture was filtered through Whatman GF/B filters (wetted with PBS, 0.1% BSA) using a Filtermate 196 harvester (Packard). Filters were then washed twice with PBS, 0.1% BSA and 40 μL scintillator was added to each well before the bound radioactivity was measured in a TopCount devise (Perkin Elmer). Non-specific binding was assessed by adding an excess of the reference ligand to the reaction mixture. Finally IC₅₀ values were calculated with the help of appropriate analysis software.

TABLE 1
Binding affinity of compounds towards
human AD brain homogenate (ADH)

| Compound | IC₅₀ human ADH |
|---|---|
| No. | [nM] |
| 1 | 9 |
| 2 | 17 |
| 3 | 13 |
| 4 | 18 |
| 5 | 400 |
| 6 | 5 |
| 7 | 258 |
| 10 | 4 |
| 11 | 28 |
| 12 | 26 |
| 13 | 46 |
| 14 | 140 |

### Autoradiographical Analysis

Fresh frozen as well as paraffin embedded sections of the frontal lobe from Alzheimer's dementia patients, frontotemporal dementia patients and age matched controls were used for the study.

Frozen sections, sliced at 18 μm thickness on a cryostate (Leica, Germany) and paraffin sections, sliced on a sliding microtom (Leica) at a thickness of 6 μm, were mounted onto glass slides (Superfrost Plus, Fa. Menzel, Braunschweig Germany). Frozen sections were allowed to adhere to the slides for several nights at −20° C. The paraffin sections were deparaffinized using routine histological methods. For binding studies sections were incubated with the labeled test compound at 10-20 Bq/μL diluted in 25 mM Hepes buffer, pH 7.4, 0.1% BSA (200-300 μL/slide) for 1.5 h. in a humidified chamber. For blocking experiments an excess of the unlabeled test substance was added to the incubation mixture. After hybridization, sections were washed four times with Hepes buffer, 0.1% BSA (or alternatively two times with 40% ethanol) and finally dipped two times into water for 10 sec. The air-dried sections were exposed to imaging plates and signals were detected by a phosphoimager device (Fuji BAS5000).

### Biodistribution

Biodistribution and excretion studies were performed in male NMRI mice (body weight app. 30 g; 3 animals per time point). The animals were kept under normal laboratory conditions at a temperature of 22±2° C. and a dark/light rhythm of 12 h. Food and water were provided ad libitium. During an acclimation period of at least 3 days before the beginning of the study animals were clinically examined to ascertain the absence of abnormal clinical signs.

At 2 min, 5 min, 30 min, 1 h, 4 h post intravenous injection via the tail vein of ca. 185 kBq of the test compound, urine and feces were quantitatively collected. At the same time points, animals were sacrificed by decapitation under isoflurane anaesthesia and organs and tissues of interest were removed for the determination of radioactivity using a gamma-counter. For analysis the decay corrected percentage of the injected dose per tissue weight (% ID/g±standard deviation) was calculated.

TABLE 2
Brain uptake and brain wash-out of compound 28 expressed
as percentage of injected dose per gram tissue [% ID/g].

| | | | Brain wash-out |
|---|---|---|---|
| | Brain uptake | Brain uptake | ratio |
| Compound | at 2 min | at 30 min | [% ID/g at 2 min]/ |
| (Example) | [% ID/g] | [% ID/g] | [% ID/g at 30 min] |
| 28 | 7.66 | 0.78 | 9.8 |

The [¹⁸F] signal was detected at 2 min and 30 min after compound administration to mice. Note the favourable high brain uptake and fast wash-out from healthy mouse brain, which is devoid of plaques.

The tracer 28 shows an advantageous rapid elimination of the radioactive background signal from the brain.

The studies described above indicate that radiolabeled compounds of the invention are useful as imaging agents for amyloid beta plaques. They can penetrate the intact blood-brain barrier and bind specifically to amyloid beta deposits.

1. A Compound of formula I 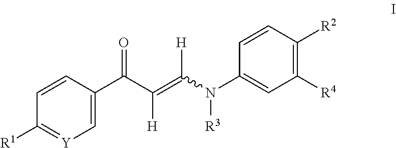
for use as diagnostic agent or imaging agent for imaging diseases associated with beta amyloid deposits
wherein
R¹ is H, phenyl or X;
R² is H, OCH₃, N(CH₃)₂, O—(CH₂)ₙ—X; (CH₂)ₙ—X or X_{;}
R³ is H;
R⁴ is H or Br;
X is F or ¹⁸F_{;}
Y is CH, CCN, CCF₃ or N; and
n is 1, 2 or 3;
with the proviso that one of the substituents R¹ and R² comprises ¹⁸F or is ¹⁸F,
or an isomeric form of said compound;
and pharmaceutically acceptable salts and salt complexes thereof.

2. A pharmaceutical diagnostic or imaging agent composition comprising a compound of claim 1 and a pharmaceutically acceptable excipient.

3. A method of diagnosing or treating a disease associated with beta amyloid deposits comprising administering a compound of claim 1.

4. The method of claim 3, wherein the disease is Alzheimer's disease.

5. The method of claim 3, wherein the disease is amyloidosis.
